(19) United States
(12) Patent Application Publication
(10) Pub. No.: US 2006/0029634 A1
(43) Pub. Date: Feb. 9, 2006

(54) Porous structures

(76) Inventors: Michael C. Berg, Cambridge, MA (US); Hyunku Ahn, Asan-City (KR); Lei Zhai, Somerville, MA (US); Robert E. Cohen, Jamaica Plain, MA (US); Michael F. Rubner, Westfield, MA (US)

Correspondence Address: STEPTOE & JOHNSON LLP, 1330 CONNECTICUT AVENUE, N.W., WASHINGTON, DC, 20036, US

(21) Appl. No.: 10/912,540

(22) Filed: Aug. 6, 2004

(51) Int. Cl. A61F 13/00

(52) U.S. Cl. 424/422

(57) ABSTRACT

A polyelectrolyte multilayer can be deposited on a surface and converted to a porous structure by aqueous processing. The porous structure can be loaded with a compound.

### FEDERALLY SPONSORED RESEARCH OR DEVELOPMENT

The U.S. Government may have certain rights in this invention pursuant to Grant No. 94-00224 awarded by MIT MRSEC and Grant No. 972-02-1-0016 awarded by DARPA.

### TECHNICAL FIELD

This invention relates to porous structures.

### BACKGROUND

Alternating layers of oppositely charged polyelectrolytes can be deposited on a surface. A suitable aqueous treatment can convert the deposited polyelectrolytes to a porous structure. The degree of porosity can be controlled by selection of the treatment conditions.

### SUMMARY

A porous polyelectrolyte multilayer can be loaded with a compound, for example, a drug, a liquid crystal, or ionic liquid. Compounds loaded in the multilayer can alter its properties, such as optical and electrical properties. The compound can also be released from the multilayer. The rate of release can be controlled, for example by controlling the porosity of the multilayer. The multilayer can include porous and non-porous regions.

Porous regions of polyelectrolyte multilayers can have pore sizes ranging from 40 nm to 20 μm. The porous structures can be optically transparent in the visible range, such as when pore size is restricted to values smaller than 150 nm. The pore morphology can be locked in by crosslinking the film using heat or chemistry. These porous films can be loaded with a variety of materials for different applications. The loading technique can be used for any kind of molecule.

The ability to introduce porosity selectively within specific regions of a polymer has further extended the utility of porous polyelectrolyte structures as photonic structures and as optical sensors. The thickness, pore size and level of porosity of spatially distinct regions within a solid polymer matrix can be controlled. This control can be particularly important in the fabrication of heterostructures having a complex arrangement of nanoporous regions. Complex arrangements can be useful in, for example, a Fabry-Perot micro-cavity (see, for example, Yariv, A. An Introduction to Theory and Applications of Quantum Mechanics Wiley: New York, 1982, which is incorporated by reference in its entirety). The layer-by-layer processing schemes developed for the manipulation of polyelectrolytes are ideally suited for this task.

A release device can include a porous polyelectrolyte multilayer. The release device can be a thin polymer film that can be coated onto virtually any surface. This makes it possible to coat implants and deliver therapeutics locally. It also adds the ability to study the release of a drug from the surface onto which a cell is attached. Most drug delivery systems are capsules or gels rather than coatings of surfaces making them incapable for use in this manner.

In general, a porous polyelectrolyte multilayer can be used in, for example, optical structures, optical devices, and drug delivery devices. The optical structures can reflect a desired wavelength of light and transmit other wavelengths. An optical device can alternate between transparent and opaque states, acting as a light shutter. A drug delivery device can change color as the drug is depleted from the device.

In one aspect, a polymer-containing structure includes a layer over a substrate, wherein the layer includes a first polymer region and a second polymer region, where the first polymer region responds to a greater extent than the second polymer region when exposed to a pore-altering medium. The first region can include a first polyelectrolyte and a second polyelectrolyte. The second region can include a third polyelectrolyte and a fourth polyelectrolyte. The first region can be disposed between the substrate and the second region. The second region can be disposed between the substrate and the first region. The second region can remain substantially non-porous when exposed to a pore-altering medium. The first region can be porous and the second region can be substantially non-porous. The first region can include a nanopore. The first region can be substantially free of micropores. The first region can become less porous when exposed to a pore-closing medium. The first region can include a compound. The compound can be a drug, a liquid crystal, or an ionic liquid. The substrate can be glass, plastic or metal.

In another aspect, a polymer-containing structure includes a plurality of alternating fixed and variable regions disposed on a substrate, where the variable regions respond to a greater extent than the fixed regions when exposed to a pore-altering medium. A variable region can include a first polyelectrolyte and a second polyelectrolyte. A fixed region can include a third polyelectrolyte and a fourth polyelectrolyte. The variable regions can each independently have a thickness less than 1000 nm. The fixed regions can each independently have a thickness less than 1000 nm. The alternating fixed and variable regions are arranged as a series of alternating layers over the substrate. The thicknesses and refractive indices of the variable regions and the thicknesses and refractive indices of the fixed regions can be selected such that the structure reflects a predetermined wavelength of light. The structure can be substantially free of pores having a diameter of 150 micrometers or greater. The thickness of a variable region can change upon exposure to a pore-altering medium. The refractive index of a variable region can change upon exposure to a pore-altering medium.

In another aspect, a polymer-containing structure includes a first polymer region and a second polymer region, wherein the first polymer region has a refractive index that can be altered by an aqueous treatment of the structure. The aqueous treatment can alter the porosity of the first region. The aqueous treatment need not substantially alter the porosity of the second region. The aqueous treatment can increase or decrease the porosity of the first region.

In another aspect, a drug delivery device includes a plurality of alternating porous and substantially non-porous regions disposed on a substrate. The device can include a compound distributed in a porous region. A porous region can include a first polyelectrolyte and a second polyelectrolyte. A substantially non-porous region can include a third polyelectrolyte and a fourth polyelectrolyte. The plurality of alternating porous and substantially non-porous regions can be arranged as a series of alternating layers over the substrate. The alternating layers of the series can each independently have a thickness and a refractive index selected such that the device reflects light of a predetermined wavelength. The device can include a delivery medium distributed in a porous region. The delivery medium can alter the refractive index of the porous region, thereby altering the reflection of light of a predetermined wavelength. The delivery medium can include a drug. The device can change color when the delivery medium exits the porous region.

In yet another aspect, an optical device includes a porous polymeric structure arranged on a substrate and a liquid crystal distributed in the pores of the polymeric structure. The porous polymeric structure can form a layer over the substrate. The device can include electrode arranged to apply an electric field across the layer when a voltage is applied to the electrodes. Applying an electric field across the layer can increase transmission of light through the porous polymeric structure.

The details of one or more embodiments are set forth in the accompanying drawings and the description below. Other features, objects, and advantages will be apparent from the description and drawings, and from the claims.

### BRIEF DESCRIPTION OF THE DRAWINGS

FIGS. 1A and 1B are graphs depicting thickness measurements of polyelectrolyte multi layers.

FIG. 2 is a graph depicting thickness measurements of polyelectrolyte multilayers.

FIG. 3 depicts atomic force microscopy images of a polyelectrolyte multilayer.

FIGS. 4A and 4B are scanning electron microscopy images of a polyelectrolyte multi layer.

FIG. 5 is a graph depicting thickness measurements of a polyelectrolyte multilayer.

FIG. 6 depicts scanning electron microscopy images of polyelectrolyte multilayers.

FIG. 7 is a photograph of a microscope slide coated with a porous structure and immersed in a liquid crystal.

FIG. 8 is a graph depicting contact angles of liquids on a porous surface.

FIG. 9 is a graph depicting transmission spectra of an optical shutter.

FIG. 10 is a graph depicting transmission spectra of an optical shutter.

FIG. 11 is a graph depicting transmission through an optical shutter.

FIG. 12 is a graph depicting transmission through an optical shutter.

FIG. 13 is a graph depicting infrared difference spectra of an optical shutter.

FIG. 14 is a graph depicting optical contrast ratios of optical shutters.

FIGS. 15A and 15B are atomic force microscopy images of porous surfaces.

FIG. 16 is a graph depicting thickness measurements of a polyelectrolyte multilayer.

FIG. 17 is a graph depicting thickness measurements of a polyelectrolyte multilayer.

FIG. 18 is a graph depicting reflectance spectra of polyelectrolyte multilayers.

FIG. 19 is a graph depicting refractive indices of polyelectrolyte multilayers.

FIG. 20 depicts the structure of polyelectrolyte multilayers, and corresponding reflectance spectra.

FIG. 21 depicts the structure of a polyelectrolyte multilayer, and corresponding reflectance spectra.

FIG. 22 depicts the structure of a polyelectrolyte multilayer, and corresponding reflectance spectra.

FIG. 23 is a graph depicting reflectance spectra of a polyelectrolyte multilayer. The inset is a graph depicting thickness measurements of the polyelectrolyte multilayer.

FIG. 24 is a graph depicting reflectance spectra of a polyelectrolyte multilayer.

FIG. 25 is a graph depicting transmission spectra of a polyelectrolyte multilayer.

FIG. 26 is a graph depicting reflectance spectra of a polyelectrolyte multilayer.

FIG. 27 is a graph depicting absorbance spectra of solution exposed to a porous structure loaded with a drug.

FIGS. 28a and 28b are photomicrographs of cells exposed to a porous structure.

### DETAILED DESCRIPTION

Nano- and microporous polymers are useful in applications including separation technologies, catalyst surfaces and supports, and antireflection coatings. See, for example, He, T., et al., J. Appl. Poly. Sci. 2003, 87, 2151; Peterson, D. S., et al. J. Anal. Chem. 2003, 75, 5328; Ling, F. H., et al., J. Org. Chem. 2002, 67, 1993; Lakshmi, B. B. and Martin, C. R. Nature 1997, 388, 758; Gu, W., et al. Chem. Mater. 2001, 13, 1949; Deng, H, et al., J. Am. Chem. Soc. 1998, 120, 3522; Chen, Y., et al. J. Am. Chem. Soc. 2000, 122, 10472; Deleuze, H. et al., Polymer 1998, 39, 6109; Sundell, M. J., et al., Chem. Mater. 1993, 5, 372; Ruckenstein, E. and Hong, L. Chem. Mater. 1992, 4, 122; Walheim, S. et al. Science 1999, 283, 520; and Hiller, J., et al., Nature Materials 2002, 11, 59, each of which is incorporated by reference in its entirety.

Other applications include drug delivery systems, tissue engineering, and templates for the growth of nanoscale materials (see, for example, Adiga, S. P. and Brenner, D. W. Nano Lett. 2002, 2, 567; Jang, J.-H.; and Shea, L. D. Journal of Controlled Release 2003, 86, 157; Sohier, J., et al., Journal of Controlled Release 2003, 87, 57; Yang, F., et al., Biomaterials 2004, 25, 1891; Shastri, V. P., et al., Biomaterials 2003, 24, 3133; Lin, A. S. P., et al., Biomaterials 2003, 24, 481; Misner, M. J.; et al., Adv. Mater. 2003, 15, 221; Ulrich, R.; et al., Adv. Mater. 1999, 11, 141; Yi, D. K.; and Kim, D.-Y. Nano Lett. 2003, 3, 207; and Jiang, P.; et al., Science 2001, 291, 453, each of which is incorporated by reference in its entirety).

Porous polymer systems have been prepared by a number of different techniques such as phase separation, the selective dissolution of polymer blends, the degradation of block copolymers, and the polymerization of monomers in sacrificial colloidal or nanoporous silica templates. See, for example, Zhang, G.; et al., Langmuir 2003, 19, 2434; Lin, Z.; et al., Adv. Mater. 2002, 14, 1373; Kiefer, J.; et al., Macromolecules 1996, 29, 4158; Zalusky, A. S.; et al., J. Am. Chem. Soc. 2002, 124, 12761; Urbas, A. M.; et al., Adv. Mater. 2002, 14, 1850; Xu, T.; et al., Polymer 2001, 42, 9091; Takeoka, Y.; and Watanabe, M. Langmuir 2002, 18, 5977; Park, S. H.; and Xia, Y. Adv. Mater. 1998, 10, 1045; Jiang, P.; et al., J. Am. Chem. Soc., 1999, 121, 11630; Holtz, J. H.; and Asher, S. A. Nature, 1997, 389, 829; and Li, Y. Y.; et al., Science, 2003, 229, 2045; each of which is incorporated by reference in its entirety. A polyelectrolyte has a backbone with a plurality of charged functional groups attached to the backbone. A polyelectrolyte can be polycationic or polyanionic. A polycation has a backbone with a plurality of positively charged functional groups attached to the backbone, for example poly(allylamine hydrochloride). A polyanion has a backbone with a plurality of negatively charged functional groups attached to the backbone, such as sulfonated polystyrene (SPS), polyacrylic acid, or a salt thereof. Some polyelectrolytes can lose their charge (i.e., become electrically neutral) depending on conditions such as pH. Some polyelectrolytes, such as copolymers, can include both polycationic segments and polyanionic segments.

Layer-by-layer processing of polyelectrolyte multilayers can be utilized to fabricate conformal thin film coatings with molecular level control over film thickness and chemistry. Charged polyelectrolytes can be assembled in a layer-by-layer fashion. In other words, positively- and negatively-charged polyelectrolytes can be alternately deposited on a substrate. One method of depositing the polyelectrolytes is to contact the substrate with an aqueous solution of polyelectrolyte at an appropriate pH. The pH can be chosen such that the polyelectrolyte is partially or weakly charged. The multilayer can be described by the number of bilayers it includes, a bilayer being the structure formed by the ordered application of oppositely charged polyelectrolytes. For example, a multilayer having the structure PAH-PAA-PAH-PAA-PAH-PAA would be said to be made of three bilayers.

These methods can provide a new level of molecular control over the deposition process by simply adjusting the pH of the processing solutions. The nonporous polyelectrolyte multilayers can form porous thin film structures induced by a simple acidic, aqueous process. Tuning of this porosity process, including the manipulation of such parameters as salt concentration (i.e., ionic strength), temperature, pH, or surfactant chemistry, can lead to the creation of micropores, nanopores, or a combination thereof. A nanopore has a diameter of less than 150 nm, for example, between 1 and 120 nm or between 10 and 100 nm. The nanopores can have diameters of less than 100 nm. A micropore has a diameter of greater than 200 nm. Selection of porosity-inducing conditions can provide control over the porosity of the coating. For example, the coating can be a nanoporous coating, substantially free of micropores. Alternatively, the coating can be a microporous coating, having pores with diameters of greater than 200 nm, such as 250 nm, 500 nm, 1 micron, 2 microns, 5 microns, 10 microns, or larger.

The porosity of polyelectrolyte multilayer can be changed by contacting the polyelectrolyte multilayer with a pore-altering medium. A pore-altering medium can be a pore-forming medium that can induce the formation of pores or increase the size of existing pores in a polyelectrolyte multilayer. Alternatively, a pore-altering medium can be a pore-closing medium that can induce the closing of pores or reduce the size of existing pores in a polyelectrolyte multilayer.

The properties of weakly charged polyelectrolytes can be precisely controlled by changes in pH. See, for example, G. Decher, Science 1997, 277, 1232; Mendelsohn et al., Langmuir 2000, 16, 5017; Fery et al., Langmuir 2001, 17, 3779; Shiratori et al., Macromolecules 2000, 33, 4213; and U.S. patent application Ser. No. 10/393,360, each of which is incorporated by reference in its entirety. A coating of this type can be applied to any surface amenable to the water based layer-by-layer (LbL) adsorption process used to construct these polyelectrolyte multilayers. Because the water based process can deposit polyelectrolytes wherever the aqueous solution contacts a surface, even the inside surfaces of complex objects can be coated.

The fabrication of polyelectrolyte multilayer (PEM) thin films is a simple technique for creating various thin film optoelectronic devices and nanostructured thin film coatings. See, for example, Wu, A., et al., J. Am. Chem. Soc. 1999, 121, 4883; Onitsuka, O., et al., Biosens. Bioelectron. 1994, 9, 677; Decher, G. and Schlenoff, J. B., Multilayer Thin Films: Sequential Assembly of Nanocomposite Materials, Vch Verlagsgesellschaft Mbh, 2003; and Decher, G, Science 1997, 277, 1232, each of which is incorporated by reference in its entirety. The layer by layer (LbL) deposition process provides a means to create multilayer films one molecular layer at a time. Although the polyelectrolytes are sequentially adsorbed during LbL assembly, the internal structure of the assembled multilayer is highly interpenetrated (see, for example, Mendelsohn, J. D., et al., Langmuir 2000, 16, 5017, which is incorporated by reference in its entirety). The multilayer assembly is driven by electrostatic interactions between the oppositely charged polyelectrolytes. LbL systems, for example poly(acrylic acid) (PAA) and poly(allylamine hydrochloride) (PAH), can provide control over the physical and chemical state of the assembled films. Parameters such as charge density, bilayer thickness, molecular conformation and the degree of the interchain ionic bonding can be varied (see, for example, Park, S. Y., et al., Langmuir 2002, 18, 9600, which is incorporated by reference in its entirety).

The pores can be loaded with drug molecules or electrically responsive molecules. In the former case, changes in the optical properties of the dielectric mirror can be used to monitor the loading and/or release of biomaterials such as DNA or drugs as has been demonstrated in porous silicon based reflectors. See, for example, Chan, S.; et al., Mater. Sci. Eng. C 2001, 15, 277; and Lin, V. S.-Y.; et al., Science 1997, 278, 840, each of which is incorporated by reference in its entirety. Using multilayers, the rate of release of a drug through the heterostructure can be controlled by the choice of barrier layers, their thickness and the sequence in the heterostructure. Liquid crystals loaded into the pores provide a means to vary the refractive index of the porous PAH/PAA blocks with application of an electric field, thereby producing a dynamically tunable Bragg reflector.

Polymer-dispersed liquid crystal (PDLC) films are most often prepared by emulsion or phase separation procedures in which droplet morphology is poorly controlled (see, for example, Kitzerow, H.-S., Liq. Cryst., 1994, 16, 1, which is incorporated by reference in its entirety). Most liquid crystals are organic, rod-like shaped molecules, several nanometers in length and a few angstroms in diameter. Because of this elongated shape and polarizability, the liquid crystal molecule has an anisotropic refractive index. The optical anisotropy of liquid crystal molecules is the basis for LC displays. The macroscopic properties of the liquid crystals can be changed readily by external stimuli. A strong molecular dipole moment can allow the long axis of the molecule to align with an applied field. In the voltage-off state, the refractive index of each liquid crystal domain depends on the domain size (see, for example, Park, N.-H., et al., Colloid Polym Sci., 2001, 279, 1082; and Ren, H-W. and Wu, S-T. Applied Physics Letter, 2002, 81(19), 3537, each of which is incorporated by reference in its entirety). As the voltage increases, the refractive index of each domain decreases and the size dependent profile of the refractive indices is flattened. When the LC molecules are ordered, liquid crystal domains exhibit different refractive indices along the parallel and perpendicular axes (see, for example, Fergason, J. L. Scientific American 1964, 211, 77; Kahn, F. J. Physics Today 1982, 70, 66; and Macrelli, G. Solar Energy Materials and Solar Cells, 1995, 39, 123). Using Mie theory for guidance, the refractive index mismatch, concentration, shape and particle size can all influence the amount of light scattering observed (see Morrison, I. D.; Ross, S. Colloidal Dispersions, Wiley-Interscience, 2002; Chapter 1). An increase in diameter from 50 nm to 2 μm results in a six order of magnitude increase in the scattered-light intensity. Size distribution effects are also observed. PDLC devices with LC droplets of 2-3 μm diameters offer the best electro-optical properties, owing to the balance between transmittance and scattering of incident light by LC droplets of that size (see Decher, G., Science 1997, 277, 1232).

A microporous film can be created simply by immersing multilayer thin films of the weak polyelectrolytes PAA and PAH in an acidic solution (pH 2.4) for 2-24 hrs and rinsing with deionized water (pH=5.5) for about 15 seconds (see, for example, Hiller J., et al., Nature Materials 2002, 1, 59; and Mendelsohn, J. D., et al., Langmuir 2000, 16, 5017, each of which is incorporated by reference in its entirety). These microporous films can be loaded with the liquid crystal mixtures E7 and BL037, resulting in tunable optical materials. A light shutter is one application of the electrically induced transmittance effect exhibited by the LC-loaded microporous films.

Microporosity in polyelectrolyte multilayers can be introduced by a low pH solution treatment. See Mendelsohn, J. D.; et al., Langmuir 2000, 16, 5017, which is incorporated by reference in its entirety. A number of other strategies have been developed for creating both micro- and nanoporous multilayer films (see, for example, Kim, B. Y.; and Bruening, M. L. Langmuir 2003, 19, 94; and Fu, Y.; et al., Macromolecules 2002, 35, 9451; each of which is incorporated by reference in its entirety). Certain multilayer assemblies based on weak polyelectrolytes, such as, e.g., poly(acrylic acid) (PAA) and poly(allylamine hydrochloride) (PAH), can undergo a phase separation process at low pH that produces either micro- or nanoporous material depending on the treatment conditions. In addition, these transitions to the porous state can be completely and repeatedly reversed by treatment at a higher pH. A pH-gated change in the degree of ionization of the PAA chains can drive these transitions.

It is possible to fabricate polyelectrolyte multilayer heterostructures including multiple nanoporous and solid regions. The spatial arrangement and thickness of different regions can be controlled at the molecular level through the layer-by-layer dipping process (see, for example, Wang, T. C.; et al., Adv. Mater. 2002, 14, 1534, which is incorporated by reference in its entirety). The nanopores can be opened and closed by using simple pH treatments. Reversible pH-gated nanoporosity transitions can be induced in polyelectrolyte multilayers. The multilayers can include a non-pore-forming region. The non-pore-forming region can be adjacent a pore-forming region. Multiple non-pore-forming regions can be adjacent to a pore-forming region. Reversible porosity transitions can occur in a pore-forming region when it is sandwiched between non-pore-forming regions. The nanopores of complex thin film heterostructures can be filled with non-ionic small molecules through a simple infiltration process. These structures can be useful in, for example, tunable dielectric mirrors (Bragg reflectors), electro-optical shutters, pH-gated dielectric mirrors, vapor sensors, and drug delivery systems.

### EXAMPLE 1

Poly(acrylic acid) (PAA) (MW=90,000 g/mol) was obtained from Polysciences as a 25 wt % aqueous solution and poly(allylamine hydrochloride) (PAH) (MW=70,000 g/mol) from Aldrich Chemicals. Polyelectrolyte deposition baths were prepared at 10⁻² M (based on the repeat unit molecular weight) using 18 MΩ Millipore water whose pH was adjusted with 1M HCl or NaOH. The liquid crystal materials E7 (nₒ=1.52, nₑ=1.74), a eutectic mixture of cyanobiphenyls and a cyanoterphenyl, and BL037 (nₒ=1.52, nₑ=1.81), 4-pentyl-4′-cyanobiphenyl, were purchased from Merck Ltd. All chemicals were used without further purification.

The multilayer thin films were deposited via an automated dipping procedure using an HMS programmable slide stainer from Zeiss, Inc. Clean indium tin oxide coated glass slides (ITO, surface resistivity 20 Ω) were immersed in a polyelectrolyte solution (PAH first, pH 8.5) for 15 minutes followed by rinsing in three successive baths of water (pH=5.5) with light agitation, for 2, 1, and 1 minutes, respectively. The substrates were immersed into the oppositely charged PAA polyelectrolyte solution (pH 3.5) for 15 minutes and subjected to the same rinsing procedure. After the desired number of layers had been deposited on ITO-coated substrates, the substrates were blown dry with air, and placed in an 80° C. oven for 1 hour. The polyelectrolyte multilayer film on the side of the glass not coated with ITO was removed by 1.0 M HCl solution. To make the microporous structure, multilayer substrates were immersed in an acidic solution (pH 2.4) for 2-24 hrs, rinsed with deionized water (pH=5.5) for about 15 seconds, blown dry and then oven dried at 80° C. for 1 hour. Total pore volumes were estimated from measurements of the thickness increase of the film. Thickness measurements were performed on dried films by profilometry. For surface characterization, an atomic force microscope (Digital Instruments Dimension 3000 Scanning Probe Microscope, Santa Barbara, Calif.) was used in tapping mode. Scanning electron microscopy was performed on a JEOL 6320 FESEM with an acceleration voltage 5 kV. For cross section imaging, the microporous multilayer film on the glass slide was scored and fractured at room temperature. The fracture surface of the polymer film was coated with 100 Å Au/Pd by vapor deposition.

The microporous voids were filled with nematic liquid crystal through capillary action. The liquid crystal was allowed to fill the microporous structure for 30 minutes. The LC-loaded devices were heated at 120° C. oven for 2 hours to crosslink the polyelectrolyte multi layer.

UV-visible measurements were performed with an Oriel Model 68806 power supply with a 75 W arc lamp and multispec detector. Instaspec software was used to display the data. The electro-optical properties were investigated at room temperature (25° C.). The relaxation measurements were performed by monitoring transmitted light intensity of a probe light from a diode laser (630 nm). Electro-optical measurements were performed by using a combination of a function generator and an AC high voltage amplifier.

Infrared measurements using a Hewlett-Packard 4284A equipped with a Nicolet spectrometer were performed to examine the electrodynamic responses of the liquid crystalline material devices to perturbations imposed by an electric field. A spectrum of the E7-filled device at 0 V was used as the background and the field response data were computed and presented as the difference spectra. The spectral resolution of the scans was 4 cm⁻¹ and the spectral range was limited to 2200-3500 cm⁻¹ due to the absorbance of indium tin oxide. To apply a voltage across the device, a DC power supply with an output range of 0-100 V was used.

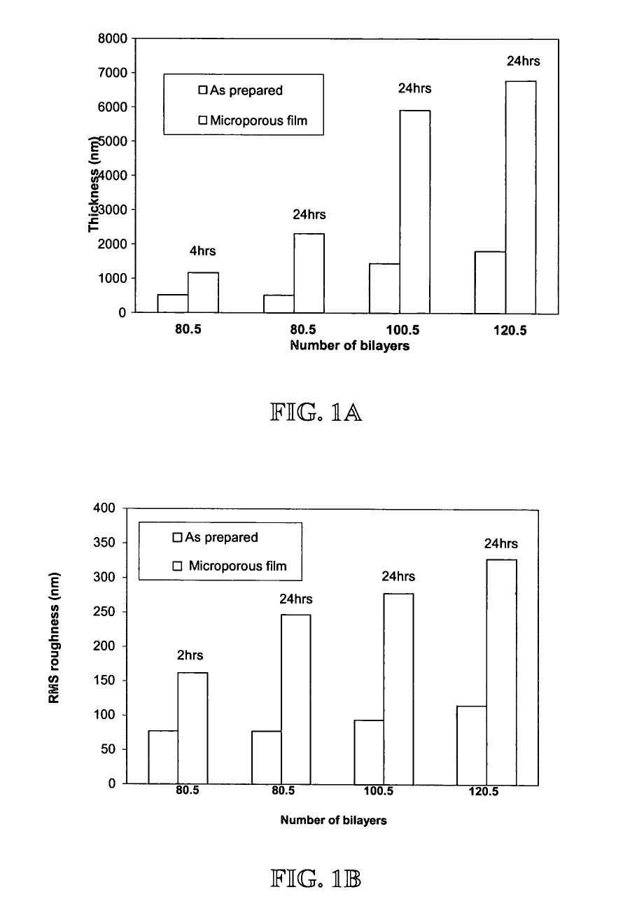

PAH/PAA multilayer films were deposited under the conditions of pH 8.5 for the PAH deposition bath and pH 3.5 for the PAA deposition bath (denoted as 8.5/3.5 PAH/PAA). Under these conditions, 8.5/3.5 PAH/PAA multilayers form with weakly ionized PAA chains depositing onto nearly fully charged PAH surfaces. The molecular organization of these 8.5/3.5 PAH/PAA multilayers can be manipulated to produce a microporous morphology (see Mendelsohn, J. D. et al., Langmuir 2000, 16, 5017). The transition is induced by exposing the film to acidic aqueous solutions at pH 2.4. The low-pH environment of the transformation bath can cleave interchain ionic bonds by the protonation of carboxylate groups, allowing localized reorganization and phase separation of PAH/PAA multilayer films from the acidic water. The films increased in thickness upon exposure to low pH. This thickness change was coupled to the emergence of the highly porous structure. Longer exposure to acidic conditions can lead to greater porosity and larger pores. The thickness continued to increase with acid exposure time up to 24 hours. The dry thicknesses of the porous multilayers were in the range 1.0-6.0 μm, where the corresponding fully dense precursors were 0.3 to 1.8 μm thick. The RMS roughness of the acid-treated films was similarly quite high, reaching up to 350 nm for a 120.5 bilayer film compared to a roughness value of 115 nm in the untreated film. FIGS. 1A and 1B show thickness and roughness data for multilayers at different numbers of bilayers, measured by profilometry before and after exposure to the acidic water and rinse in the deionized water. A single bilayer was formed from a sequential deposition of PAH at pH 8.5 and PAA at pH 3.5; non-integer values indicate that the structure is capped with a final PAH deposition.

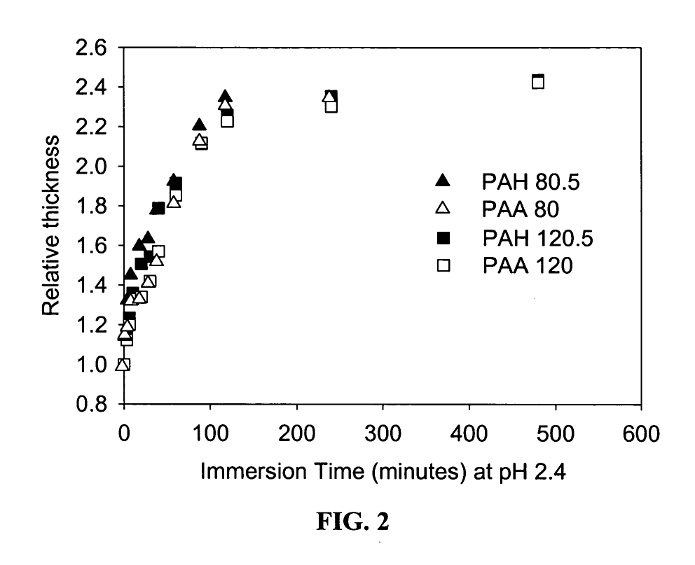

FIG. 2 illustrates the outermost layer effect and time effect when 8.5/3.5 PAH/PAA films were immersed into pH 2.4 and subsequently rinsed with pH 5.5 water for 15 seconds. Comparing the PAA-topped and PAH-topped films, a difference in the thickness of the porous structures was observed for films treated in acid for shorter exposure times. This effect disappears when longer acid treatments are employed, such as longer than 4 hours.

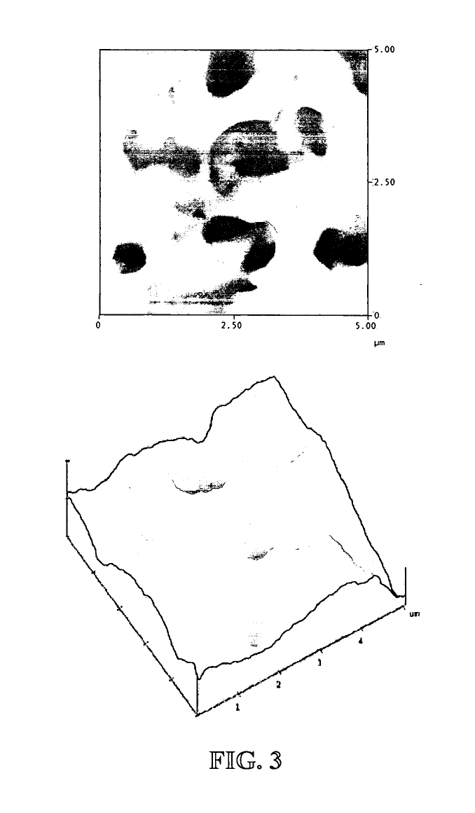

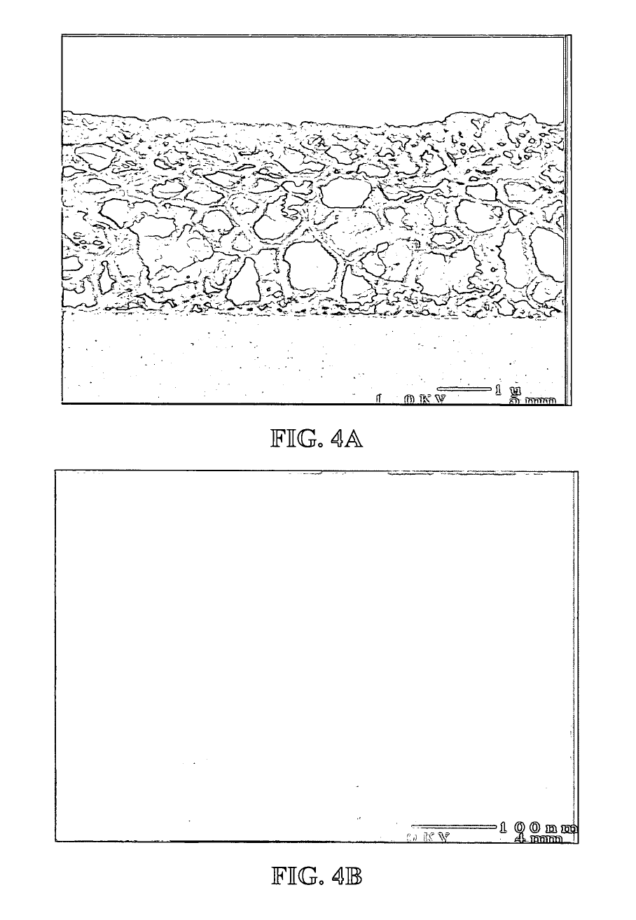

FIG. 3 shows tapping mode atomic force microscopy (AFM) images for a 8.5/3.5 PAH/PAA 80.5 bilayer film after it was immersed for 2 hours in an aqueous bath of pH 2.4, rinsed in water for 15 seconds, and dried. A highly interconnected, porous microstructure was observed with a characteristic pore length scale in the range of 1-2 μm. The resulting microporous films were stable in air. A sideview SEM image of a 120.5 bilayer film that was initially exposed for 4 hours to a pH 2.4 bath and rinsed for 15 seconds in water and dried is presented in FIG. 4, which shows clearly that a porous structure was formed in the multilayer. The internal porous structure was highly interconnected. The average pore size was about 0.8-1.2 μm.

The stable microporous structure revealed in FIG. 4 collapsed rapidly upon re-immersion in a low pH aqueous environment. Fixed negative charges on the walls of the pores can provide repulsive forces strong enough to stabilize the structure that develops in the original, fully dense PEM when treated at pH 2.4 and rinsed at pH 5.5 (see Hiller J. et al., Nature Materials 2002, 1, 59). An example of this phenomenon found in nature is the cowpea chlorotic mottle virus (see, for example, Speir, J. A., et al., Structure 1995, 3, 63; and Douglas, T., and Young, M., Nature 1998, 393, 6681, each of which is incorporated by reference in its entirety). Reprotonation at low pH of negatively charged carboxylate ions on the walls of the micropores can lead to the collapse of the microporous structures.

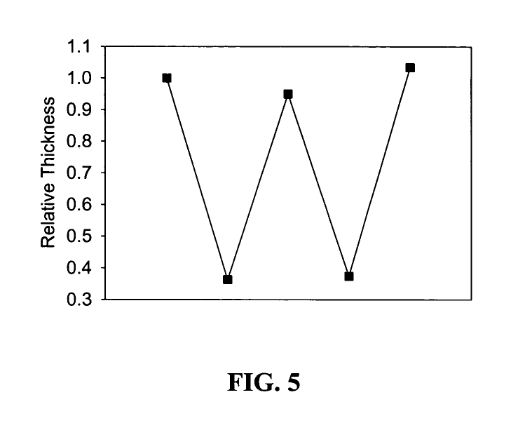

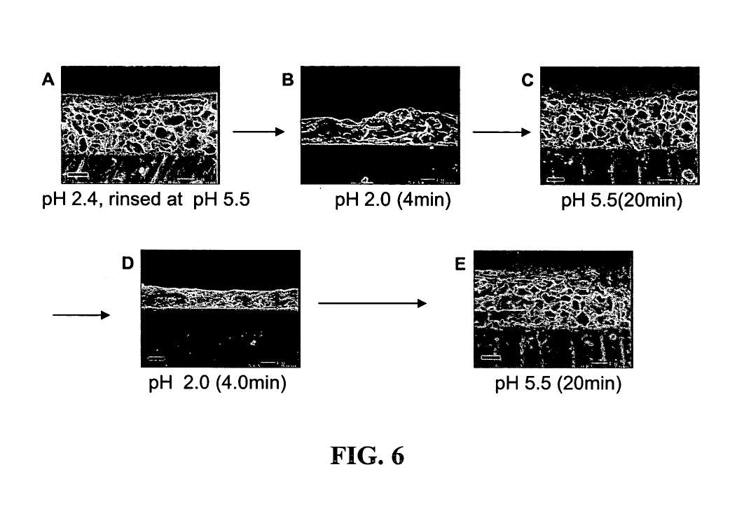

Conformal thin coatings thus can be reversibly cycled between fully dense and microporous states. As shown in FIGS. 5 and 6, the dry thickness of an initially microporous film changed reversibly as the film was cycled between aqueous baths at pH 2.0 and 5.5 and dried. The reversible transition produced a thickness change of nearly a factor of three for this 120.5 bilayer film. The SEM images in FIG. 6 indicate that the reversible transition did not lead to fully reconsolidated bulk films; in other words, some internal interfaces did not fully heal in the cyclic treatment. This residual internal structure influenced the optical properties of the film. It was possible to open and collapse the microporous structure by controlling the pH.

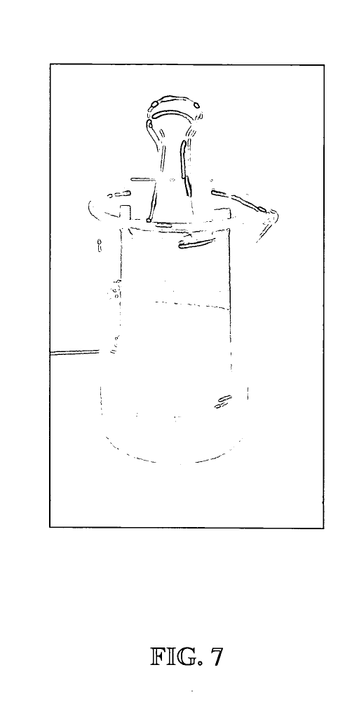

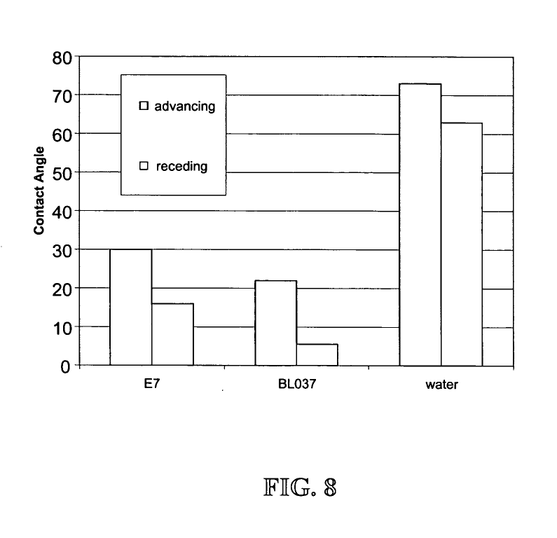

The microporous devices were filled with nematic liquid crystal by capillary action (see FIG. 7). The wicking action was facilitated by the favorable wetting characteristics of the LC molecules. As shown in FIG. 8, the contact angles of BL037 (advancing 21°, receding 7°) and E7 (advancing 30°, receding 16°) were smaller than those of water on the microporous multilayer film. The average isotropic refractive index (nᵢₛₒ=(nₒ+2nₑ)/3) of the LC droplets was larger than that of polymer matrix (see, for example, Ren, H-W. and Wu, S-T. Applied Physics Letter, 2002, 81(19), 3537, which is incorporated by reference in its entirety). The nₒ and nₑ are defined as ordinary refractive index and extraordinary refractive index, respectively. These values are nₒ=1.53, nₒ=1.81 for BL037 and nₒ=1.52 and nₑ=1.75 for E7. Accordingly, the values of the average isotropic refractive index of BL037 and E7 are 1.71 and 1.67, respectively. The measured refractive index of the polymer matrix was 1.52.

Configuration of LC molecules in liquid crystal domains depends on the size and shape of the domains, surface anchoring, and applied fields. Alignment of the LC molecules along an applied electric field decreased the refractive index in that direction to a value close to that of the polymer matrix. This refractive index matching in the on state induced transparency in otherwise opaque films. Strong scattering in the off state can give rise to higher optical contrast. Off state scattering was increased by increases in nᵢₛₒ of the LC, the film thickness, the volume fraction of LC domains, and optimizing the domain size relative to the wavelength of incident light. Both AC and DC voltage can operate the devices. AC can be preferable for minimizing electrochemical degradation of the LC.

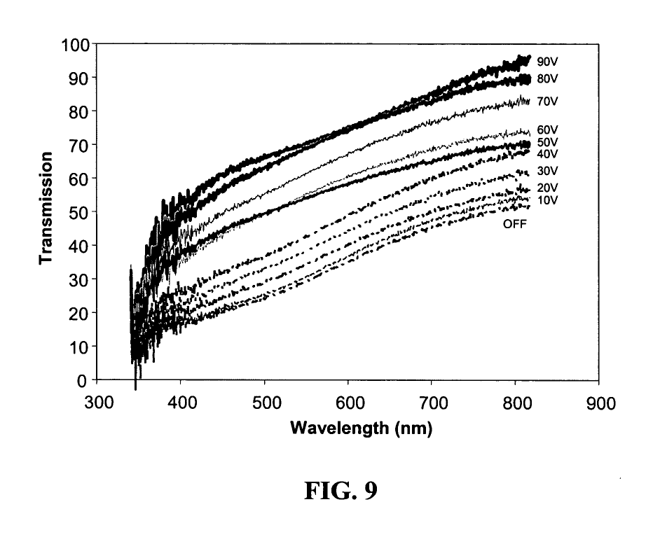

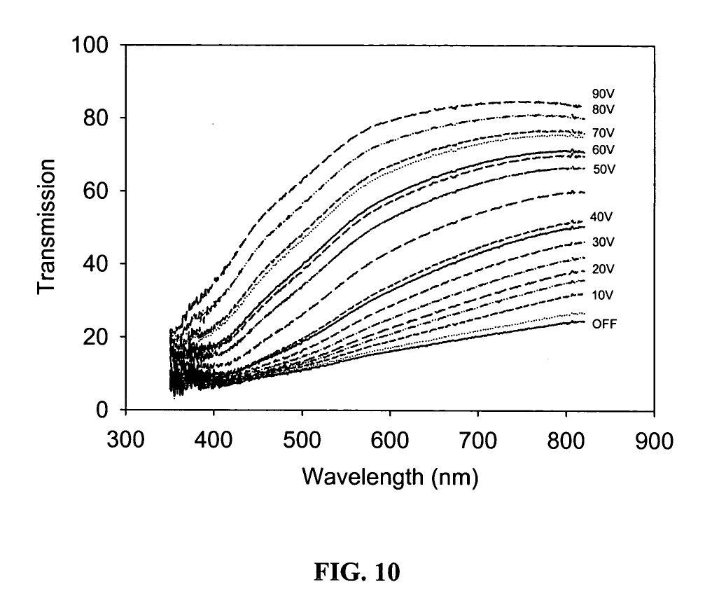

Measurements of optical transmission through the films as a function of applied electric field were recorded. FIG. 9 shows the transmission spectra of an E7-filled 8.5/3.5 PAH/PAA 161 bilayer device at different applied DC voltages from 0 V (off state) to 90 V. FIG. 10 presents transmission spectra for a BL037-filled 161 bilayer device. The BL037-filled 8.5/3.5 PAH/PAA 161 bilayer device had stronger scattering (i.e. is more opaque in the off state) than a device having the same multilayer structure but filled with E7. The enhanced opacity can be due to the larger value of nᵢₛₒ for BL037. Increasing film thickness favors multiple scattering. Greater optical contrast was observed for thicker devices.

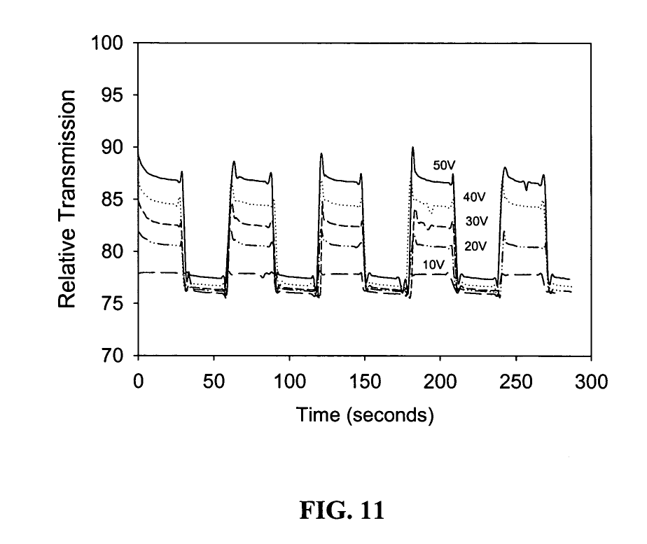

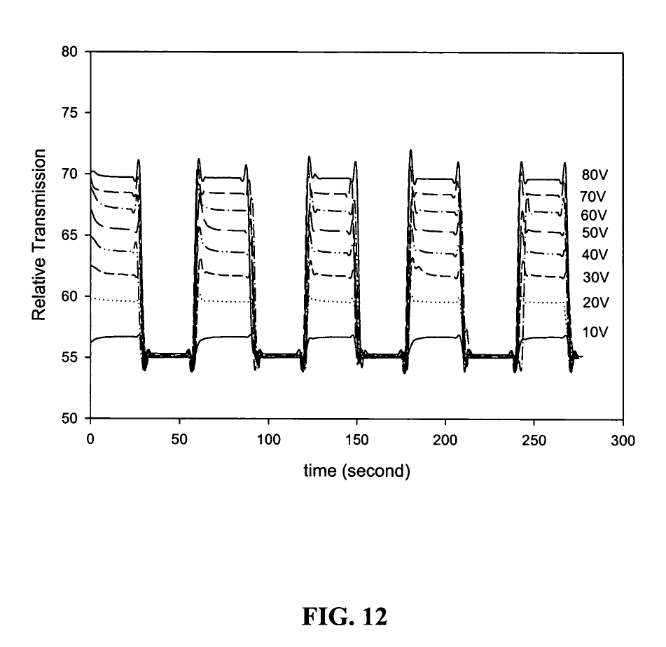

FIGS. 11 and 12 show the changes in relative transmittance at 630 nm as a function of the applied voltage for an E7-filled 161 bilayer device and a BL037-filled 161 bilayer device, respectively. The intensity of incident light measured with no device in place was used to normalize the measured transmission intensities of the devices. The results of FIGS. 11 and 12 show that the switching is rapid and reversible. The closed (off state) shutter based on BL037 is more effective than the E7 device at blocking incident light (32% vs. 16%) due to significantly larger value of isotropic refractive index which makes the embedded BL037 LC pores more potent scattering center (□n =0.19 for BL037 and 0.15 for E7 where □n is defined as the difference between the isotropic refractive index of the LC (nᵢₛₒ) and the polymer refractive index). When the liquid crystal domains fully orient in the electric field, their refractive index change may bypass the precise index-matching condition with the polymer matrix. This could account for the overshoot in recorded transmission intensity that accompanied the on-off switching. The decay time was investigated after removing a DC electric field of 50V applied to an E7 filled 161 bilayer device and a BL037 filled 161 bilayer device, respectively. The decay time is less than 1 second for the E7 filled device and approximately twice as long for the BL037 filled device. Thinner devices relax more rapidly when the same electric field is applied.

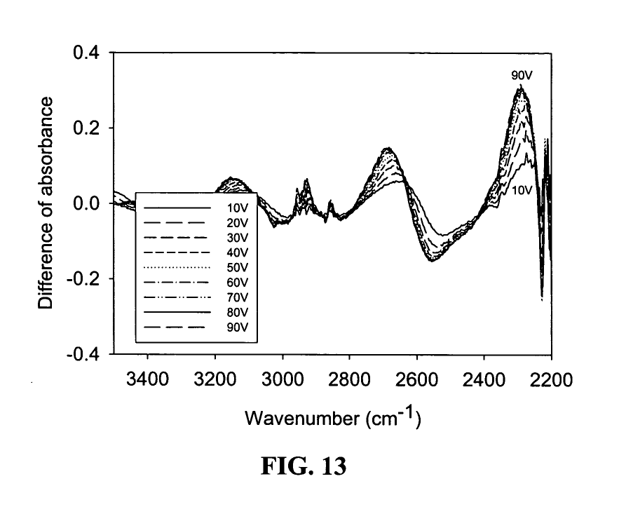

An E7-filled 241 bilayer microporous device was sandwiched between two ITO electrodes and studied using FTIR spectroscopy. This measurement was used to examine the electrodynamic responses of liquid crystal domains to perturbations imposed by an electric field. The IR beam was incident in a direction parallel to that of the applied electric field and thus the liquid crystal oriented along the same direction as the propagation of the incident IR beam. A series of difference spectra, measured as a function of the applied potential, is shown in FIG. 13. The dynamics and organization of the molecules showed perturbations that appear to originate from a complex interplay of surface and confinement effects. Starting at a voltage of zero and increasing by 10 V increments up to 90 V, spectra of the device were acquired and the difference spectra were determined against the spectrum measured with the device held at 0 V. FIG. 13 presents the difference spectra for the CN stretching mode (centered at 2226 cm⁻¹ at 0 V) at successive voltage steps. Vibrational modes interact with light polarized parallel to the transition moment direction. The CN stretch has a large vector component along the long axis of molecule and it orients along the lines of the applied field (see, for example, Sakamoto, K., et al., Vib. Spectrosc., 1999, 19, 61; Noble, A. R., et al., J. Am. Chem. Soc., 2000, 122, 3917; and Noble, A. R., et al., J. Am. Chem. Soc., 2002, 124, 15020; each of which is incorporated by reference in its entirety).

Optical contrast ratio (CR) is often used for describing the usefulness of optical shutters. The contrast ratio is defined as CR=Tₒₙ/T_{off}, where Tₒₙ and T_{off} stand for the integrated transmittance in the range of 350 nm ˜800 nm with and without external electric field, respectively.

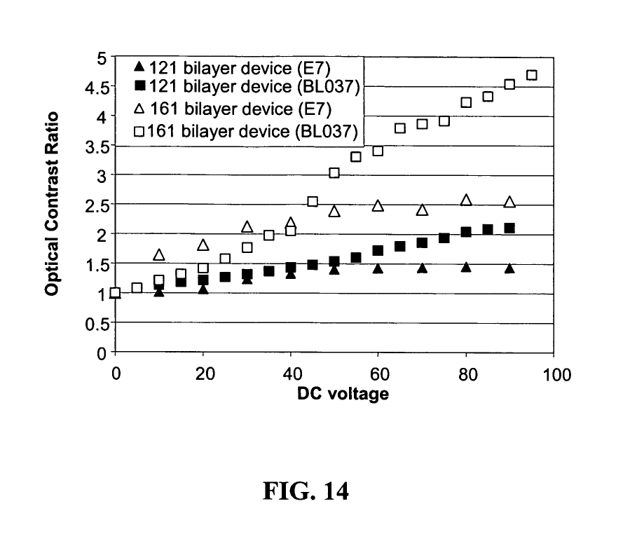

Light scattering from the device can increase with the film thickness because of higher effective concentration of LC and the higher probability of multiple scattering. Light scattering can be highest when the LC domain size approaches the wavelength of the incident light. FIG. 14 shows the optical contrast ratios with different liquid crystal molecules for two device thicknesses. The BL037 filled 161 bilayer 8.5/3.5 PAH/PAA device shows a higher optical contrast ratio than the E7 filled 161 bilayer 8.5/3.5 PAH/PAA device. E7 filled devices appear to saturate near 50 V. From comparison of BL 037 and E7 at the same electric field strength, the BL 037 device has 30% higher optical contrast ratio than the E7-based device at this field strength. The optical contrast ratios of PMMA/BL037 device and PVA/BL037 PDLC device among PDLC devices were reported as 8 and 12, respectively (see, for example, Klosowicz, S. J. and Zmija, J. Optical Engineering 1995, 34(12), 3440, which is incorporated by reference in its entirety). Comparing with these typical PDLC device, the low optical contrast ratios of our devices are from different droplet size, shape and density, and the LC and polymer refractive indices and wavelength.

### EXAMPLE 2

PAH (M_{w}=70,000) and SPS (M_{w}=70,000) were obtained from Sigma-Aldrich (St. Louis, Mo.). PAA (M_{w}=90,000) was obtained from Polysciences (Warrington, Pa.). All the chemicals were used as received. Deionized water (>18 M□ cm, Millipore Milli-Q), with an unadjusted pH of approximately 5.5, was exclusively used in all aqueous solutions and rinsing procedures. Liquid crystal E7 was purchased form EM Industries, Inc.

Polyelectrolyte multilayers (PEMs) were assembled on glass microscope slides, 3-aminopropyltriethoxysilane coated microscope slides (LabScientific, Inc.) or polished single-crystal silicon wafers (<1100>) using an automated Zeiss HMS slide stainer as previously described (see Shiratori, S. S.; and Rubner, M. F. Macromolecules 2000, 33, 4213, which is incorporated by reference in its entirety). Silicon wafers and glass substrates were degreased in a detergent solution followed by deionized water rinses prior to multilayer assembly. Silane coated microscope slides were used as received.

PAH/PAA blocks were built using pH 8.5 (±0.01) PAH (10⁻² M by repeat unit) and pH 3.5 (±0.01) PAA (10⁻² M) aqueous solutions which were pH adjusted by using either 1 M NaOH or 1 M HCl. Briefly, PEMs on glass slides and silicon wafers were formed by first immersing substrates into the PAH solution for 15 minutes followed by one 2 minute and two 1 minute immersions into water as rinsing steps. Then, the substrates were immersed into the PAA solution for 15 minutes followed by identical rinsing steps. The adsorption and rinsing steps were repeated until desired numbers of bilayers were obtained. One bilayer is defined as a single adsorption of a polycation followed by an adsorption of a polyanion; thus a half-integer number of bilayers of PAH/PAA ends with PAH as the outermost layer.

Thickness was measured using ellipsometry (Gaertner) from films on silicon wafers at wavelengths of 633 nm and from films on silicon wafers and glass by profilometry (Tencor P10). AFM characterization (Digital Instruments Dimension 3000 Scanning Probe Microscope) was performed in tapping mode with Si cantilevers. Near-normal reflectivity (fixed 7° off normal to the plane of the film) from one side of a substrate was measured on a Cary SE ultraviolet/visible/near-infrared spectrophotometer. The film on the other side of the substrate was removed and replaced with a black backing material.

The refractive index of the porous PAH/PAA blocks with air or liquid filled pores was estimated using an effective medium approximation (see, for example, Romanov, S. G.; et al., Physical Review E 1999, 63, 056603; and Sareni, B.; et al., J. Appl. Phys. 1996, 80, 1688, each of which is incorporated by reference in its entirety).
n_{f}=Vₚ*n₁+(1−Vₚ)*nₛ Eq. 1
Vₚ=(H−H₀)/H Eq. 2

In Eq. 1, n_{f} is the effective value of refractive index for the air- or liquid-filled, porous PAH/PAA blocks; n₁ is the known refractive index of the air or infiltrated liquid, and ns is the refractive index of a fully dense PAH/PAA block. Vp is the pore faction of the porous PAH/PAA block. H₀ and H are the block thickness before and after porosity transitions, respectively. This approximation was used to obtain an effective refractive index of the PAH/PAA blocks in Bragg reflectors and vapor sensors. Index values calculated this way compare favorably to those obtained by means of simulation (within 5% or better).

Theoretical reflectivity responses (simulations) were calculated using the transfer matrix method attributed to Abeles (see Abeles, F.; Ann. Phys. 1950, 5, 596, which is incorporated by reference in its entirety). The optical response of an interference filter is dependent upon the refractive index and thickness of each region in the structure. In the simulations, the thicknesses of both the porous and non-porous regions, as well as the refractive index of the non-porous regions were known. Because the experimental reflectance data revealed the location of the maximum Bragg reflectance, the Bragg Equation could be used to solve for the unknown refractive index of the porous regions. In order to produce the theoretical spectral responses in this paper, the matrix method was implemented in MATLAB. As described by Hecht, the thicknesses and refractive index data were used to create a unique 2 by 2 matrix for each region in the structure (see Hecht, E. Optics; 4th ed.; Pearson Addison Wesley: Reading, Mass., 2001, which is incorporated by reference in its entirety). These 2 by 2 matrices were subsequently multiplied together in the order they appeared to create a total transfer matrix for the entire structure. A straightforward equation was then used to derive theoretical reflectance values from the entries of the total transfer matrix. Because the entries of each matrix were dependent upon the wavelength of the incident light, the above procedure was repeated multiple times over a range of wavelengths to give theoretical reflectance vs. wavelength plots.

In the experiments on vapor detection, a cross-linked film was put into a closed quartz spectrophotometer cell with air inside. The transmittance spectrum was obtained on a Cary 5E ultraviolet/visible/near-infrared spectrophotometer. The film was then put into a closed quartz spectrophotometer cell with a saturated analyte vapor. The transmittance of the film was measured at the wavelength of the reflection band until it reached an asymptotic upper limit. Both the film and the spectrophotometer cell were dried in vacuum before use with another analyte.

Polyelectrolyte multilayers constructed from PAH and PAA can exhibit reversible, pH-driven morphological reorganizations leading to the formation of micro- and nanoporous films. For example, multilayers assembled with the PAH dipping solution at a pH of 7.5 and the PAA dipping solution at a pH of 3.5 (7.5/3.5 PAH/PAA) form micropores after a brief exposure to a pH 2.4 aqueous solution, while 8.5/3.5 PAH/PAA multilayers form nanopores after immersion in a pH 1.8 aqueous solution. In both cases, a final brief rinse in water completes the porosity transformation. Multilayer heterostructures having porosity forming regions sandwiched between (or constrained by) non-porosity forming regions can be made. The pore-forming regions can include PAH/PAA bilayers, and the non-pore-forming regions can include PAH/sulfonated polystyrene (SPS) bilayers.

An 8.5 bilayer of 8.5/3.5 PAH/PAA structure was surface capped with 25 bilayers of PAH/SPS (denoted as (PAH/PAA)_{8.5}-(PAH/SPS)₂₅). Ellipsometry measurements revealed that the PAH/PAA and PAH/SPS blocks in the fully dense structure were 86 nm and 47 nm thick respectively, and the average refractive index of the film was 1.55. The film was immersed for 30 seconds in a pH 1.8 acidic solution followed by a 15 second rinse in deionized water (pH 5.5), conditions previously found to create nanopores in 8.5/3.5 PAH/PAA multilayers. After the treatment, multiple macroscopic cracks were observed on the film surface, most likely induced by an uncontrolled swelling of the underlying PAH/PAA block, which in turn generated fractures in the less swellable outermost PAH/SPS block. In contrast, no cracks were observed when a (PAH/PAA)_{8.5}-(PAH/SPS)₂₅ film was treated with a pH 2.2 acidic solution for one minute followed by a one minute rinse in deionized water. In this case, a more controlled swelling of the underlying PAH/PAA block eliminated the formation of cracks in the PAH/SPS surface block. After this treatment, the total dry film thickness increased from 133 to 230 nm while the average refractive index decreased from 1.55 to 1.38. During the fabrication process, the PAH/SPS block was assembled at pH 4.0 with both polymers in their fully ionized form. This bilayer system swells slightly in acidic solution and collapses back to its original thickness and refractive index (1.55) in the dry state (see, for example, Hiller, J. Ph.D. Thesis, Massachusetts Institute of Technology, 2003, which is incorporated by reference in its entirety). Thus, the change in dry thickness observed after treatment is the result of a change in the PAH/PAA block. All of these observations suggest that the film thickness change is due to the formation of pores selectively in the PAH/PAA block.

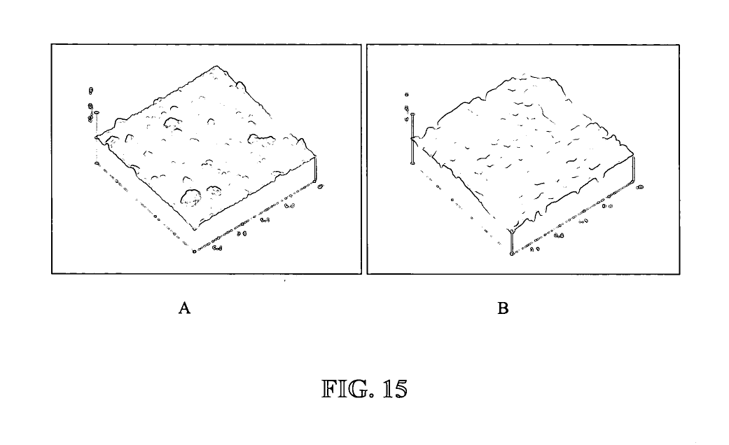

The surface of the pH 2.2 treated (PAH/PAA)_{8.5}-(PAH/SPS)₂₅ film was examined using atomic force microscopy (AFM) (FIG. 15A). The measured RMS surface roughness (14.2 nm) was lower than that of a porous 8.5-bilayer PAH/PAA film (RMS roughness 23.5 nm) formed on a silicon wafer with the same treatment (FIG. 15B), indicating that the presence of a PAH/SPS capping block results in a smoother surface after porosity development. Dimples on the order of hundreds of nanometers, however, were observed on the treated (PAH/PAA)_{8.5}-(PAH/SPS)₂₅ film. These craters resulted from the collapse of the PAH/SPS block over larger pores formed in the underlying PAH/PAA block. Large pores of similar dimension can be seen in the 8.5-bilayer PAH/PAA film after treatment (FIG. 15B). As will be mentioned later, a small fraction of pores large enough to scatter light can be formed during the porosity inducing treatment. No dimples, however, were observed on the surface of a treated film capped with a thicker PAH/SPS block ((PAH/PAA)_{8.5}-(PAH/SPS)₅₀ where the PAH/SPS block had a thickness of 112 nm.

The pH of the porosity inducing treatment and the thickness of an adjacent non-porosity forming block can influence the properties and microstructure of the final multilayer film. Thus, the treatment needed to create nanopores within confined regions of a multilayer film must be optimized for the particular heterostructure under consideration. It can be preferable to avoid the formation of micropores without destroying or disrupting the heterostructure organization by, for example, uncontrolled swelling. The number and sequence of PAH/PAA and PAH/SPS bilayers, the pH of the acid treatment solution, and the immersion time in the acid and rinse solutions can be important parameters that need to be considered.

A series of films including 1-10 bilayers of PAH/PAA sandwiched between two 50-bilayer PAH/SPS blocks was fabricated. Two different treating solutions (pH 2.3 and pH 2.2) were used to induce porosity in the PAH/PAA regions. The sandwich structures—(PAH/SPS)₅₀-(PAH/PAA)ₘ-(PAH/SPS)₅₀ (m=1, 1.5, 2, 2.5, 3, 3.5, 4, 4.5, 5, 5.5, 6, 7, 8, 9, 10)—were immersed in a pH 2.3 acidic solution for one minute followed by a one minute rinse in deionized water. The (PAH/SPS)₅₀-(PAH/PAA)₇₋₁₀-(PAH/SPS)₅₀ films turned cloudy after this treatment, suggesting that a high level of micropores was generated in heterostructures with the thickest PAH/PAA blocks (m=7, 8, 9, 10). Heterostructures with fewer than 7 PAH/PAA bilayers, on the other hand, were essentially transparent after treatment indicating that physical constraints caused by the surrounding PAH/SPS blocks can influence the length scale of porosity development in the PAH/PAA layers. Without the PAH/SPS blocks, the PAH/PAA bilayers would form micropores under these conditions.

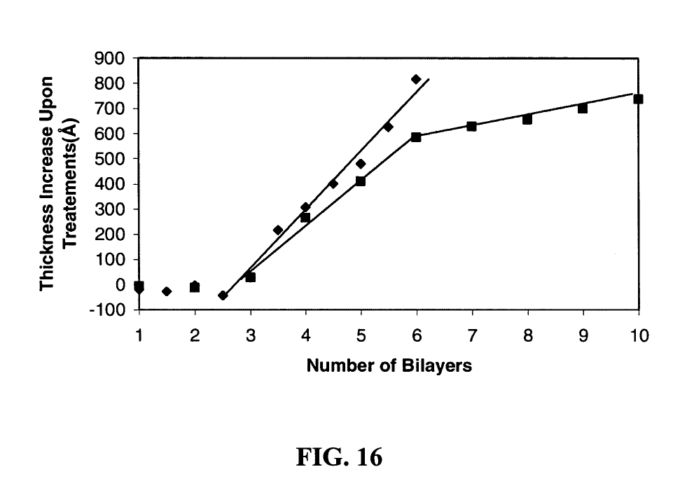

FIG. 16 (diamonds) shows the change in thickness of the PAH/PAA block as a function of the number of PAH/PAA bilayers incorporated in the heterostructure (m=1, 1.5, 2, 2.5, 3, 3.5, 4, 4.5, 5, 5.5, 6) as measured by profilometry. The thickness values were estimated with the assumption that the dry-state thickness of the PAH/SPS blocks is not influenced by the treatment. No change in thickness was observed for heterostructures containing 1 to 2.5 bilayers of PAH/PAA. Thereafter, the thickness change of the PAH/PAA block induced by treatment increases linearly with increasing number of PAH/PAA bilayers.

(PAH/SPS)₅₀-(PAH/PAA)ₘ-(PAH/SPS)₅₀ (m=1, 2, 3, 4, 5, 6, 7, 8, 9, 10) films were also treated with a one minute immersion in a pH 2.2 aqueous solution followed by a one minute water rinse. In this case, all films remained clear after the treatment suggesting that a stronger driving force for phase separation (due to a lower pH) produces a smaller fraction of micropores within the nanoporous structure. The thickness change of the PAH/PAA blocks induced by this treatment is also presented in FIG. 16 (squares). No thickness change took place in heterostructures with 1 to 2 PAH/PAA bilayers. The thickness change then increased linearly with increasing number of deposited PAH/PAA bilayers up to 6 bilayers, at which point a slope change is observed reflecting a smaller thickness change increment. The smaller thickness increment observed in the 3 to 6 PAH/PAA bilayer range for the pH 2.2 treatment compared to the pH 2.3 treatment indicates that a lower level of porosity is developed in the former case; a conclusion confirmed by refractive index results (see below). The change in slope that occurred at 6 PAH/PAA bilayers in films treated at pH 2.2 can be due to the one-minute treatment time being insufficient to activate the full porosity transition in these thicker PAH/PAA blocks. To confirm this hypothesis, a (PAH/PAA)₈-(PAH/SPS)50 multilayer film and a (PAH/PAA)₈-(PAH/SPS)₂₅ multilayer film were assembled onto silane coated glass substrates. Both films were treated with a one minute immersion in a pH 2.2 solution followed by a one minute water rinse. The thickness of the (PAH/PAA)₈-(PAH/SPS)₂₅ film increased 80 nm with this treatment whereas the thickness of the (PAH/PAA)₈-(PAH/SPS)₅₀ film increased by only 66 nm. Thus, in the same period of time, the thicker PAH/SPS capping block presents a more effective barrier to the diffusion of the treatment solution. Longer treatment times produced a larger thickness change in the film with the thicker PAH/SPS block, but also resulting the formation of light-scattering micropores. Similar results were obtained if the thickness of the PAH/PAA block increased. It can be important to optimize treatment conditions when nanopore formation is desired over micropore formation in confined PAH/PAA bilayers. In general, as the thickness of the multilayer heterostructure increases, the porosity inducing treatment must be modified appropriately. A multiple treatment protocol can be preferred, especially for very thick films.

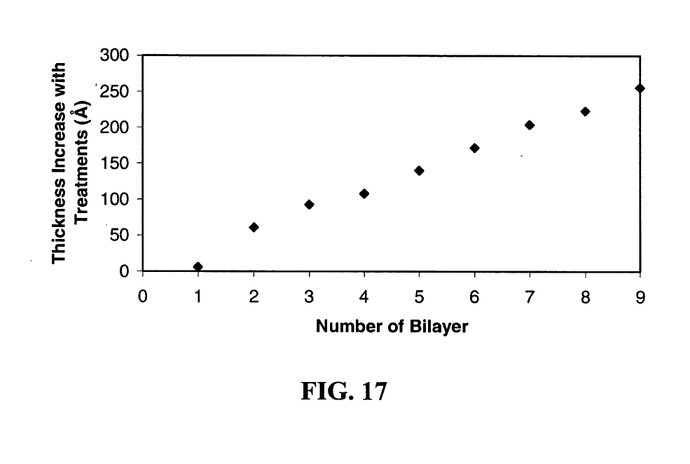

The fact that all multilayer heterostructures with 1 to 2.5 bilayers of PAH/PAA did not undergo the porosity transition indicates that interpenetration of the polymer chains nearest to the PAH/SPS blocks is preventing the phase separation process from taking place. The polymer chains in polyelectrolyte multilayers are well interpenetrated. See, for example, Baur, J. W.; et al., Langmuir 1999, 15, 6460; Joly, S.; et al., Langmuir 2000, 16, 1354; and Decher, G. Science 1997, 277, 1232, each of which is incorporated by reference in its entirety. Best estimates for PAH/PAA-PAH/SPS heterostructures are that a single polymer chain penetrates into 1 to 2 surrounding bilayers. To estimate indirectly how much interpenetration occurred at the interface of a PAH/PAA and PAH/SPS block, the thickness change of a film comprised of 1 to 9-bilayers of PAH/PAA assembled onto a PAH/SPS block with 50-bilayers was examined by using profilometry and confirmed by ellipsometry. Thickness measurements were made after a 30 second immersion in a pH 1.8 solution followed by a 15 second water rinse. A pH 1.8 acidic solution was used to ensure that only nanopore formation occurred in the PAH/PAA bilayers. As shown in FIG. 17, a thickness change (from profilometry measurements) was not observed until 2 bilayers were deposited onto the PAH/SPS block. In films with from 2 to 9 bilayers of PAH/PAA, the thickness change observed increased linearly with increasing number of PAH/PAA bilayers. These results combined with the results obtained from sandwich structures indicate that chain interpenetration sufficient to modify physical properties occurs at the level of about 1 to 2 bilayers. Thus, for a PAH/PAA block sandwiched between two PAH/SPS blocks, the top and bottom 1 to 2 PAH/PAA bilayers adjacent to the PAH/SPS blocks are restricted from undergoing the porosity transition due to chain interpenetration.

The selective introduction of nanoporosity in multilayer heterostructures can lower the refractive index of the pore-forming regions. Reflectivity measurements coupled with suitable modeling can be used to determine the refractive index of both the pore-forming and non-pore-forming regions. A multiblock stack comprised of alternating regions of high and low refractive index is the simplest example of a one-dimensional photonic structure. With suitable block thicknesses, multilayer heterostructures exhibit reflectivity bands in the visible region of the spectrum, i.e., they behave as dielectric mirrors. The optical behavior of the dielectric mirror depends upon the refractive index and thickness of each block in the structure. According to Bragg's law, the first order reflected wavelength of a film with such a periodic index profile is given as:
□₁=2(n₁d₁+n₂d₂) Eq. 3
where □₁ is the first order reflected wavelength and n₁ and n₂ are the refractive indices of the alternating blocks, and d₁ and d₂ are the and thicknesses of the alternating blocks (see Alfrey Jr, T.; et al., Polym. Eng. Sci. 1969, 9, 400, which is incorporated by reference in its entirety). The refractive index of the porous blocks can be estimated by putting experimental data, including the first order reflected wavelength of the film, the measured thickness and the known refractive index of the nonporous blocks, and the measured thickness of the porous blocks into Eq. 3. Further, a comparison of simulations of the reflectivity curves with the experimental results can be used to confirm the results of this calculation. Theoretical reflectivity curves were calculated using the transfer matrix method.

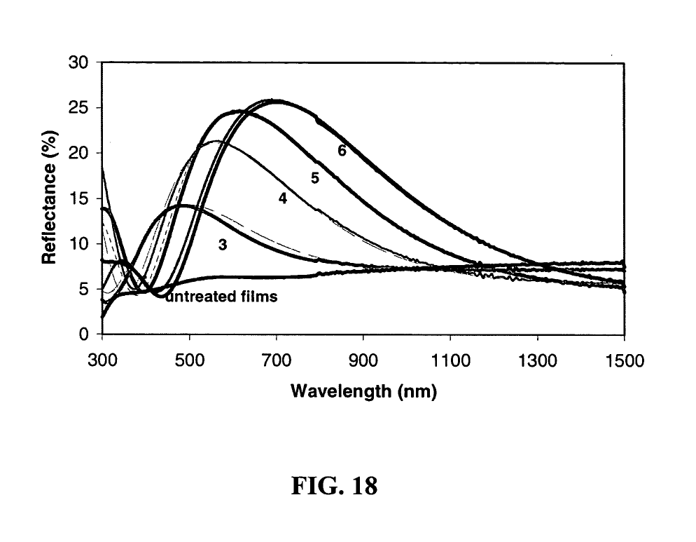

Using this approach, the refractive index of the porous PAH/PAA block in (PAH/SPS)₅₀-(PAH/PAA)ₘ-(PAH/SPS)₅₀ heterostructure films with m=3, 4, 5, 6 was calculated to be 1.24, 1.19, 1.16 and 1.15 respectively (pH 2.3 treatment) FIG. 18 shows a comparison of the measured reflectance (solid lines) of these films with the reflectance curves obtained by a simulation (dashed lines) that uses the experimentally measured thicknesses of the PAH/PAA and PAH/SPS blocks, the known refractive index of a PAH/SPS multilayer film and the above indicated refractive index values for the porous PAH/PAA block. As indicated in FIG. 18, excellent agreement was obtained between experiment and simulation.

As the number of PAH/PAA bilayers increases, the peak of the reflectance band shifts to longer wavelengths and increases in intensity. The band shift is a result of the thickness increase and index change of the PAH/PAA blocks, whereas the intensity increase is due to a decrease in the refractive index of the PAH/PAA block. The apparent refractive index of the PAH/PAA block decreased with increasing number of bilayers because of layer interpenetration that occurs at the PAH/PAA-PAH/SPS interface. The estimated refractive index of the PAH/PAA block is an average of the refractive index of the non-porous, interpenetrated PAH/PAA bilayers and the porous PAH/PAA regions. The contribution of the non-porous bilayers to this average becomes less important as the total thickness of the PAH/PAA block increases.

The combination of experimental data and theoretical simulations of the sandwich structures of FIG. 18 ((PAH/SPS)₅₀-(PAH/PAA)_{3˜6}-(PAH/SPS)50)) can be used to estimate the interpenetration thickness and the actual refractive index of the porous PAH/PAA regions. Since the interpenetration region does not undergo porosity transitions, it can be considered as a solid polymeric region extending the PAH/SPS blocks. In order to estimate the thickness of the solid and porous regions, the interpenetration thickness was added to the PAH/SPS regions and subtracted from the porous PAH/PAA regions. Simulations on the four structures were then carried out to match the experimental data. Using this approach, the best match between simulation and experiment was obtained with an average refractive index of the porous PAH/PAA regions of 1.16±0.01 and an interpenetration thickness of 4 nm, i.e., 4 nm at the top and bottom of the PAH/PAA regions did not undergo the porosity transition. This suggests that the level of porosity created in the PAH/PAA regions that actually underwent the porosity transition was about the same regardless of the number of deposited bilayers. Given that different treatments or structures may influence this interpenetration thickness value, all refractive index values for the porous PAH/PAA regions are reported as the effective values that include contributions from the interpenetrated interfacial regions and from the fully-porous zones described immediately above.

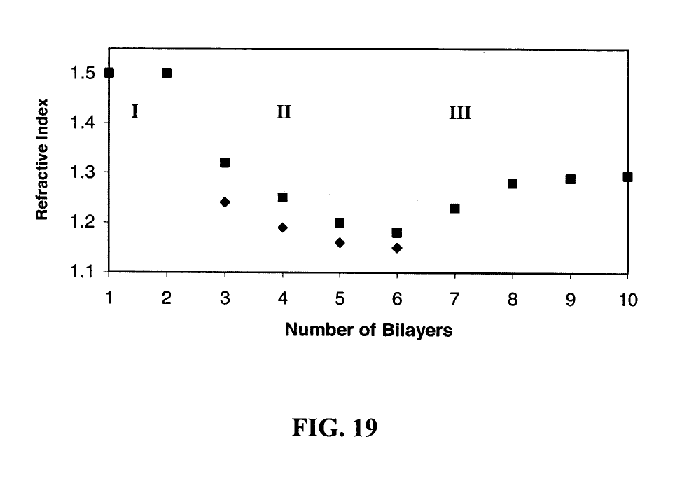

FIG. 19 presents refractive index data for the PAH/PAA block in (PAH/SPS)₅₀-(PAH/PAA)ₘ-(PAH/SPS)₅₀ films (m=1 to 6) that were treated at pH 2.3 (diamonds) and the PAH/PAA block in (PAH/SPS)₅₀-(PAH/PAA)ₘ-(PAH/SPS)₅₀ films (m=1 to 10) that were treated at pH 2.2 (squares). Refractive index values were calculated by substituting experimental data in Eq. 3 and confirmed by the agreement between simulations and the measured reflectivity curves. These data show that the porosity inducing treatment at pH 2.3 produced a PAH/PAA block with a lower refractive index than did the pH 2.2 treatment, confirming that a lower level of porosity was developed with the pH 2.2 treatment. Using this lower pH treatment, it was possible to add more PAH/PAA bilayers without the complication of excessive micropore formation. Three distinct regions can be identified in FIG. 19. In region I (1 to 2 PAH/PAA bilayers), the refractive index did not change because the development of porosity was restricted due to interpenetration at the PAH/SPS interfaces. In region II (3 to 6 PAH/PAA bilayers), the average refractive index decreased with increasing PAH/PAA block thickness as the contribution to the index by the interpenetrated PAH/PAA layers decreased. In region III (7 to 10 PAH/PAA bilayers), the refractive index increased with increasing PAH/PAA block thickness. In this case, a longer diffusion and transition time would be required to achieve a lower index due to the thickness of the PAH/PAA block. The treatment times used in this experiment produced a lower level of pore formation within thick PAH/PAA blocks (7˜10 bilayers) compared to thin PAH/PAA blocks (3˜6 bilayers), and therefore resulted in a smaller drop in the refractive index with increasing number of bilayers. Longer treatment times, however, can induce micropore formation.

Bragg Reflectors

Dielectric mirrors can be made by introducing nanoporosity controllably in select regions of a multilayer heterostructure. The dielectric mirror can have any desired optical profile over a wide range of the electromagnetic spectrum. Porosity-inducing treatment pH and time can be design factors in the fabrication of multilayer heterostructures containing multiple alternating porous and nonporous regions. For example, a multilayer heterostructure having five alternating heterostructure regions in the following sequence: (PAH/SPS)₅₀-[(PAH/PAA)₆-(PAH/SPS)₅₀]₅ was made. In this film, the thicknesses of the PAH/SPS and PAH/PAA blocks before porosity treatment were 112 nm and 61 nm, respectively. The porosity forming treatment involved immersing the film in a pH 2.3 solution for 3 minutes followed by a 3-minute water rinse and air dying. The two-step porosity formation treatment cycle was repeated three times. The final thickness of the treated film was 1257 nm. Assuming a complete and uniform transformation of the PAH/PAA regions to the nanoporous state, the thickness of each of these regions was estimated to be to 117 nm using Eq. 4:
d₁=(dₜ−N₂*d₂)/N₁ Eq. 4
where dₜ, d₁ and d₂ are the total thickness of the film, the porous PAH/PAA blocks, and the PAH/SPS blocks, respectively. N₁ and N₂ are the number of porous PAH/PAA and PAH/SPS blocks.

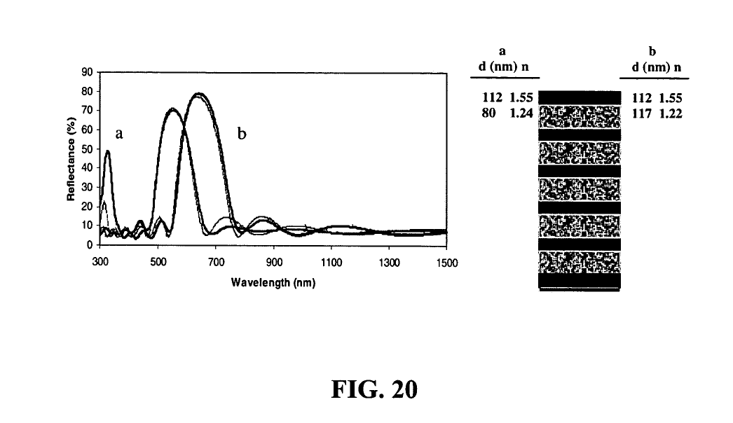

FIG. 20b shows the experimental reflectance spectrum (thick lines) of this dielectric mirror as well as the theoretical reflectance spectrum (thin lines) calculated by using the measured thickness (112 nm) and refractive index of PAH/SPS blocks (1.55) and calculated index of the porous PAH/PAA blocks (1.22, calculated using Eq. 3) and the above indicated thickness value (117 nm) for the porous PAH/PAA blocks. The simulation predicts the existence of a reflection peak at 633 nm and a maximum reflectivity of 79%. Both are in excellent agreement with the experimental results, demonstrating that with suitable treatment, dielectric mirrors with predictable optical properties can be designed and fabricated. The good agreement between the experimental data and the theoretically predicted reflectivity also suggests that each PAH/PAA region is uniformly converted into a similar state of nanoporosity (i.e., same refractive index and thickness).

The layer-by-layer processing technique allows the thickness and sequence of the various blocks present in a dielectric mirror to be easily controlled by selecting the number and types of layers deposited during assembly. See, e.g., Wang, T. C., et al., Adv. Mater. 2002, 14, 1534, which is incorporated by reference in its entirety. The position and intensity of the reflectance band (or bands) of the dielectric mirror are readily tunable by selecting the appropriate design.

FIG. 20a shows the reflectance curve of a dielectric stack fabricated with five PAH/PAA blocks: (PAH/SPS)50-[(PAH/PAA)₄-(PAH/SPS)₅₀]₅. In this case, the as-assembled thicknesses of the PAH/SPS and PAH/PAA blocks were 112 nm and 38 nm, respectively. Using the porosity treatment outlined above, the thickness of the PAH/PAA blocks increased to 80 nm and the refractive index was calculated to be 1.24 using Eq. 3. The simulation (thin line) of this dielectric mirror is again in good agreement with the experimental results (thick line). Both show the expected shift of the reflectance peak to lower wavelength (545 nm versus 633 nm). The effective medium approximation (Eq. 1 and Eq. 2,) was also used to calculate the refractive index of the porous PAH/PAA blocks in the above dielectric mirrors and predicted similar index values within 5% (results: n=1.28 for the 6-bilayer PAH/PAA block and n=1.26 for the 4-bilayer PAH/PAA block).

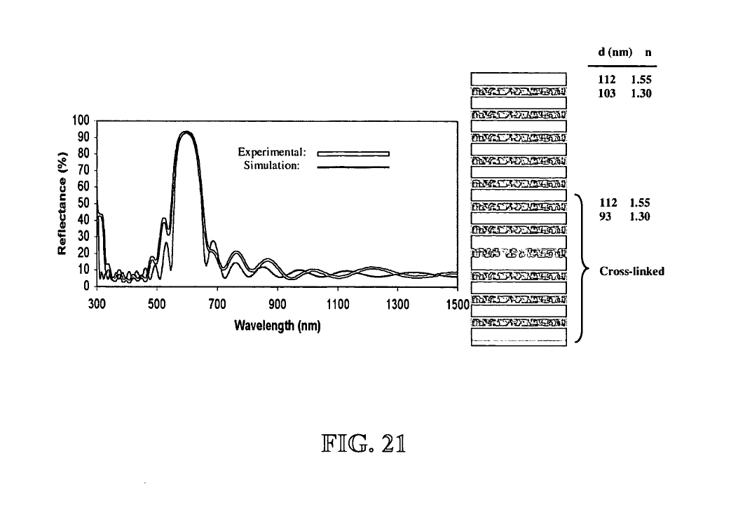

FIG. 21 shows reflectance data for a multilayer dielectric mirror with a maximum reflectance of more than 90% at 605 nm. The structure of this multilayer, (PAH/SPS)₅₀-[(PAH/PAA)₆-(PAH/SPS)₅₀]-(PAH/SPS)₅₀, contains eleven distinct 6-bilayer PAH/PAA blocks and a total of 1432 individually adsorbed polymer layers. In order to generate comparable levels of nanoporosity in all of the PAH/PAA regions within this very thick film (as assembled thickness more than 2.4 microns) about half of the structure (i.e., six alternating PAH/PAA-PAH/SPS blocks) was first assembled. Then, nanoporosity was induced in the PAH/PAA regions. The film was then thermally crosslinked (5 hours at 140° C.) to prevent further porosity development followed by addition of the remaining PAH/PAA-PAH/SPS layers needed to complete the structure. The porosity inducing treatment in both cases involved immersion in a pH 2.3 solution for 3 minutes followed by a 3-minute immersion in deionized water. This process was repeated three times. Due to crosslinking induced shrinkage, the thickness of the top five PAH/PAA blocks is about 10% larger than that of the six PAH/PAA blocks assembled in the first stage of this process. When this is taken into account, the simulation of this structure produces a reflectivity spectrum that nicely matches the experimental results (FIG. 21), demonstrating control over optical properties in a film having hundreds of layers.

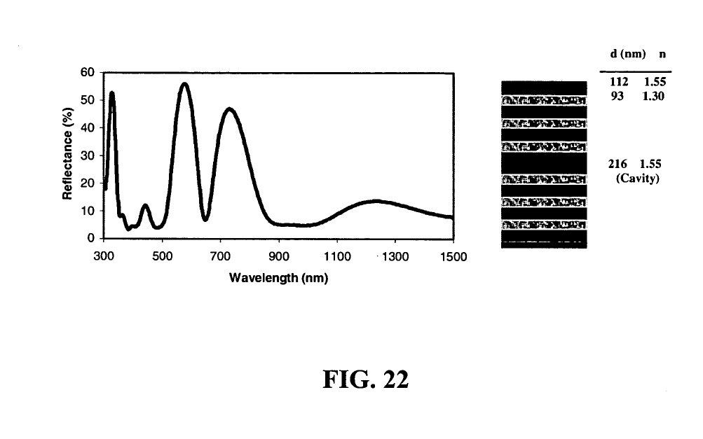

A more complex dielectric mirror having a multilayer heterostructure with a Fabry-Perot optical cavity was created (see, for example, Dirr, S., et al., Adv. Mater. 1998, 10, 167, which is incorporated by reference in its entirety). The presence of such a cavity opens up a transmission window in the reflectivity band. The cavity was created by producing a thicker PAH/SPS block in the center of the film. The structure of this film is shown in FIG. 22 along with its measured reflectivity curve. The film contained the following layer arrangement: (PAH/SPS)₅₀-[(PAH/PAA)₆-(PAH/SPS)₅₀]₂-(PAH/PAA)₆-(PAH/SPS)₁₀₀-(PAH/PAA)₆-[(PAH/SPS)₅₀-(PAH/PAA)₆]₂-(PAH/SPS)₅₀. The nanoporosity was created by using three cycles of a 3 minutes immersion in a pH 2.3 solution followed by a 3-minute immersion in deionized water. The 216 nm PAH/SPS optical cavity in this structure opens up a transmission window at 649 nm, a wavelength about twice the equivalent optical thickness of the PAH/SPS block.

Reversible pH-Gated Nanoporosity Transitions

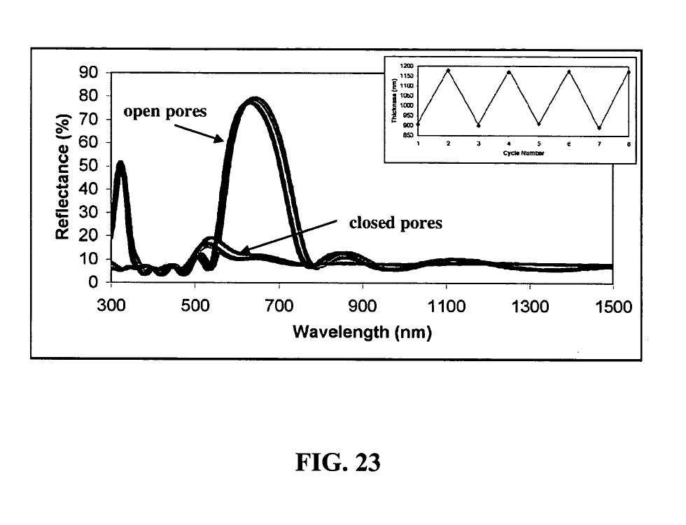

The nanoporosity transitions in 8.5/3.5 PAH/PAA multilayers are reversible. pH-Gated reversible nanoporosity transitions are also possible within confined PAH/PAA mutlilayers. A multilayer heterostructure was fabricated with 5 PAH/PAA blocks: [(PAH/PAA)₆-(PAH/SPS)₅₀]₅. As shown in the inset of FIG. 23, the dry thickness of this film changed reversibly from 900 nm to 1180 nm when it was alternately immersed for three minutes in pH 2.3 and pH 5.5 solutions, suggesting a reversible change in the thickness of the PAH/PAA block. The reflectivity data shown in FIG. 23 confirm that the origin of this reversible thickness change is the opening and closing of nanopores in the PAH/PAA blocks. In the open pore state, the film exhibits a reflectivity band with a wavelength maximum of 639 nm, whereas in the closed pore state, the film exhibits the reflection behavior close to the as-assembled, non-porous film (after pore closing, a small amount of index contrast remains between the PAH/PAA and PAH/SPS blocks (□n˜0.05). The position and intensity of the reflectivity band observed in the open pore state indicate that the PAH/PAA regions are nanoporous and have an effective refractive index of 1.23.

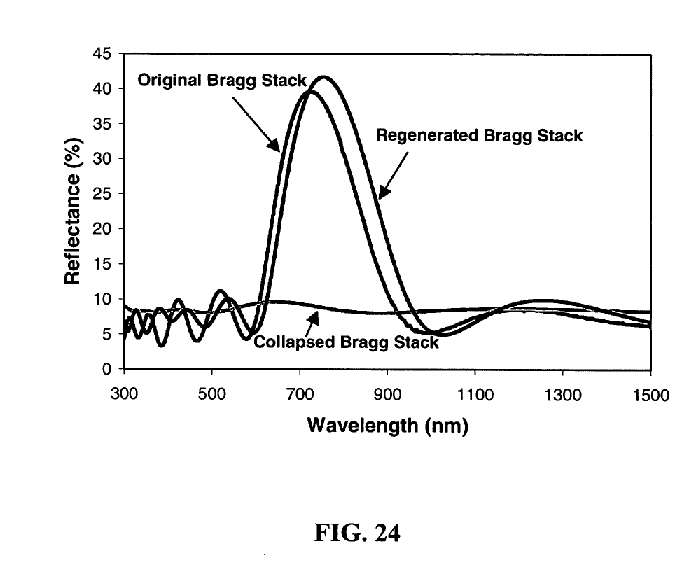

The PAH/PAA regions of these multilayer heterostructures can be cycled repeatedly between the open and closed pore state by these pH treatments. The nanopores of a nanoporous film immersed in deionized water overnight, however, were eliminated due to a further reorganization of the polymer chains. Similar behavior is observed with both microporous and nanoporous multilayers of PAH/PAA. It is possible to regenerate the nanoporous regions by treatment in a low pH solution. For example, FIG. 24 shows the reflectivity curves of a [(PAH/PAA)₈-(PAH/SPS)₅₀]₃ dielectric mirror after the overnight treatment in water and after a pH 2.2 treatment and subsequent water rinse. The overnight water treatment eliminates the Bragg reflection (trace labeled ‘Collapsed Bragg Stack’), but the reflection band is observed to reemerge after the low pH treatment (trace labeled ‘Regenerated Bragg Stack’). The reflectance band of the regenerated Bragg stack displays a slightly higher reflectance and a small shift to the red, most likely due to an additional expansion of the PAH/PAA blocks.

Applications of Multilayer Heterostructures Containing Nanoporous Domains

The optical properties of dielectric mirrors that utilize nanoporosity transitions to create the low index blocks of a Bragg stack are highly sensitive to changes in the level of porosity. A substance that penetrates into the pores can modify the refractive index of these regions and hence the optical properties. This strong coupling between optical properties and the physical or chemical state of the nanoporous regions opens up numerous possibilities in applications such as vapor sensors, monitorable drug delivery systems, or switchable Bragg gratings (see, for example, Crawford, G. P. Optics & Photonics News 2003 54; and Pikas, D. J., et al., Appl. Phys. A 2002, 74, 767, each of which is incorporated by reference in its entirety). Various kinds of Bragg stacks have been investigated for vapor sensing. Bragg stacks based on polymer replicas of nanoporous silicon films, for example, exhibit a significant reflectance-band shift upon exposure to various solvent vapors. The shift is caused by the condensation of analyte vapors within pores and the concomitant change in the refractive index of the porous regions. See, for example, Gao, J., et al., Langmuir 2002, 18, 2229, which is incorporated by reference in its entirety.

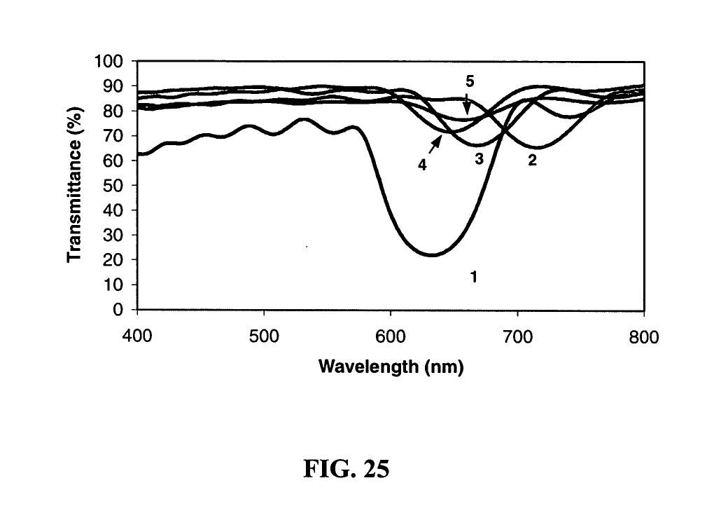

To demonstrate this effect with polyelectrolyte multilayer Bragg reflectors, a [(PAH/PAA)₈-(PAH/SPS)₅₀]₉ film was converted to the nanoporous state by alternating immersion for two minutes in a pH 2.2 and pH 9.5 solution. A higher pH rinsing solution can enhance nanopore formation in the thick PAH/PAA blocks. The film was then cross-linked by heating at 140° C. for 5 hours. The transmittance spectrum of this film is presented in FIG. 25. The transmittance spectrum of the film in air exhibited a trend of decreasing transmission with decreasing wavelength due to the presence of some light scattering, micrometer-sized pores in the PAH/PAA regions. Upon exposure to a saturated vapor of water, ethanol, acetone, or toluene, the reflectivity band decreased in intensity and shifted to longer wavelengths, leading to an increase in transmittance and a shifting of the minimum transmittance wavelength. The light scattering caused by the larger pores also diminished upon exposure of the film to these vapors. In these experiments, the same film was used for each solvent after solvent removal by air or vacuum drying. The transmittance spectrum of the dried film always returned to the curve shown in FIG. 25. These dramatic and reversible effects are a result of the change of the refractive index of the nanoporous regions that occurs when they are penetrated by condensing solvent vapor.

Table 1 presents the refractive index of each liquid analyte, and the transmission level and wavelength of minimum transmission resulting from exposure to each solvent. The transmittance was higher when the film is exposed to the vapor of a liquid with a higher refractive index. The better refractive index match between the pores filled with analyte liquid and the polymer matrix resulted in a lower film reflectance, leading to a higher transmittance. The distinct optical changes observed with different vapors can be used to distinguish analytes. Using the data generated from the toluene-exposed film, it was possible to confirm that the solvent vapor was in fact condensing into the pores. The refractive index of PAH/PAA regions with toluene filled nanopores was calculated to be 1.50 using the effective medium approximation (Eq. 1 and Eq. 2). Using this value in a reflectance simulation predicts a minimum transmission (76%) at 663 nm, which is in good agreement with the experimental result (77% at 658 nm). The more dramatic shift to the red observed with water (and somewhat with ethanol) can be due to a swelling of the multilayer by these more polar solvents.

TABLE 1

| | air | water | ethanol | acetone | toluene |
|---|---|---|---|---|---|
| Refractive index | 1.00 | 1.33 | 1.36 | 1.39 | 1.49 |
| peak transmittance (%) | 22 | 66 | 67 | 72 | 77 |
| peak wavelength (nm) | 626 | 709 | 668 | 647 | 658 |

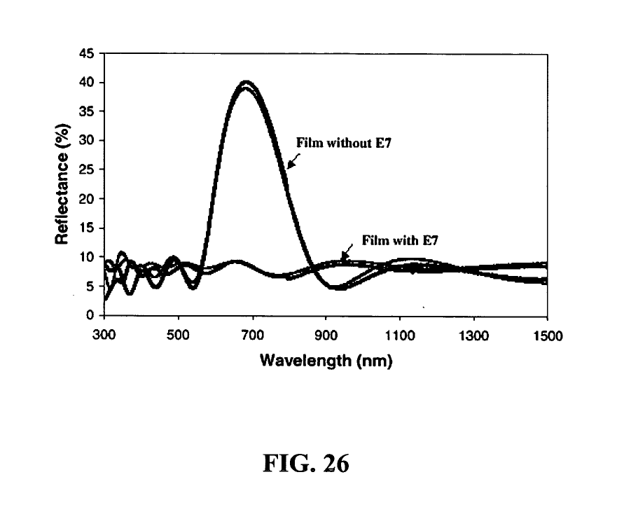

The bottom portion of a thermally crosslinked multilayer heterostructure with nanoporous PAH/PAA blocks ([(PAH/PAA)₈-(PAH/SPS)₅₀]₃) was immersed into E7 liquid crystal. After a short period of time, the liquid crystal infiltrated the pores by a wicking effect. This process could be easily monitored by observing the film under a UV lamp. As the fluorescent liquid crystal diffused through the multilayer film, the film became fluorescent. The reflectivity of the film also changed dramatically as the liquid crystal filled the pores and modified the refractive index of the PAH/PAA regions. FIG. 26 shows the change in reflectivity that occurs after the loading process. Before loading, the film behaves as a dielectric mirror with a reflectivity band centered at about 690 nm. This reflectivity band disappears when the liquid crystal is loaded into the pores.

The average/isotropic refractive index of the liquid crystal is 1.62. The average refractive index of the PAH/PAA regions therefore increases when the pores are filled, thereby reducing the index contrast between the PAH/PAA and PAH/SPS regions. The refractive index of E7-filled PAH/PAA blocks was calculated to be 1.57 using the effective medium approximation (Eq. 1 and Eq. 2) and a theoretical simulation of the reflectivity curve of the E7 loaded film (see FIG. 26) also suggests a refractive index of 1.57. The original reflectivity band was reestablished when the E7 molecules were extracted from the multilayer film using acetone. These results show that nanoporous multilayer films can be used as vehicles for loading and releasing non-ionic organic molecules.

### EXAMPLE 3

To demonstrate that non-ionic small molecules can be loaded into the porous regions of multilayer films, we utilized the well-known liquid crystal E7 (a eutectic mixture of 4-cyano-4′-n-alkylbiphenyls). To accomplish this, the bottom portion of a thermally crosslinked multilayer heterostructure ([(PAH/PAA)₈-(PAH/SPS)₅₀]₃) deposited onto a glass slide was simply immersed into the liquid crystal. After a short period of time, the liquid crystal was observed to infiltrate into the film by a wicking effect as shown in FIG. 7. This process was monitored by observing the film under a UV lamp. As the fluorescent liquid crystal diffused through the multilayer film, it became fluorescent. The reflectivity of the film also changed dramatically as the liquid crystal filled the pores and modified the refractive index of the PAH/PAA regions. Before loading, the film behaved as a dielectric mirror with a reflectivity band centered at about 690 nm. This reflectivity band disappeared when the liquid crystal was loaded into the pores.

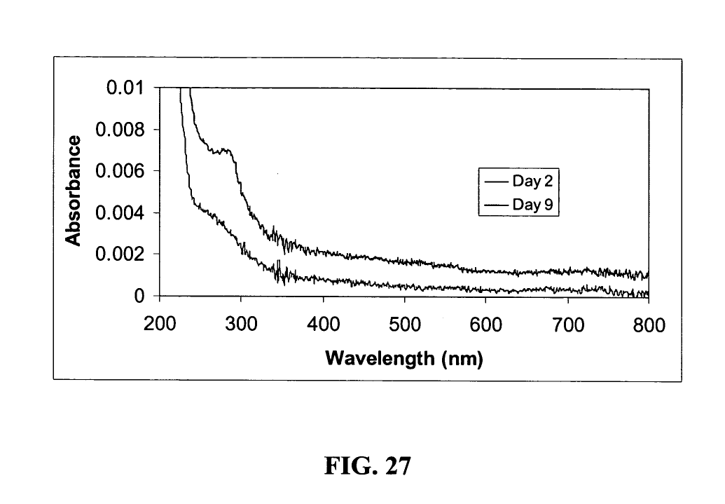

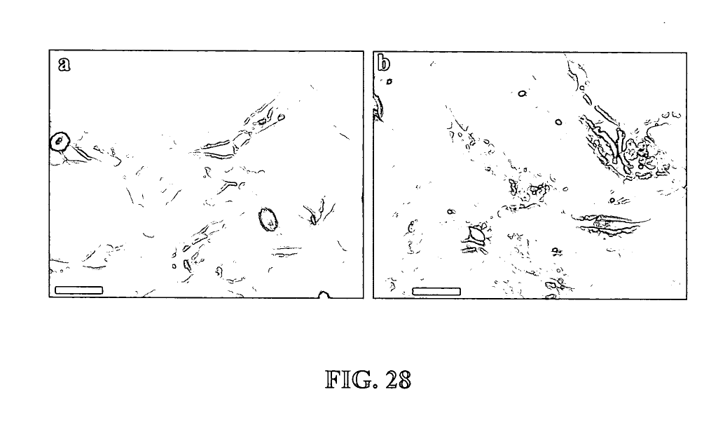

Another application of loaded porous multilayer films is controlled drug delivery. Crosslinked heterostructure-films were constructed and immersed into an organic solution containing a drug. The drug wicked up the porous layers in the film as could be monitored by the change in reflectance of the film. The controlled release of the drug in a buffer solution was observed by monitoring the absorbance change of the buffer solution. An example of these experiments is shown in FIG. 27, demonstrating the release of cytochalasin D. As can be seen from the change in peak height with time, the drug was released in a controlled manner. Cytochalasin D arrests mitosis, leaving cells with double nuclei. The control sample (FIG. 28a), which contained no drug in the pores, shows fibroblasts in their normal morphology attached to the surface. FIG. 28b shows cell culture experiments with wild type NR6 fibroblasts (nuclei stained with DAPI) indicating the cell's uptake of the drug after release from the pores. The double nuclei of the cells in FIG. 28b indicated that the cells in this sample cannot divide due to the inhibition of actin by Cytochalasin D.

Loading and Releasing of Cytochalasin D

Cytochalasin D was loaded into porous multilayer films by wicking it up in a solution of dimethyl sulfoxide (DMSO). The films consisted of alternating blocks of nonporous (poly(allyl amine hydrochloride) (PAH)/poly(sodium 4-styrene-sulfonate) (SPS)) multilayers and porous (PAH/polyacrylic acid (PAA)) multilayers. A PAH/SPS block consisting of 101 layers was built first on the substrate (PAH as the first layer). The substrate consisted of either a glass microscope slide or silicon wafer. For deposition, the pH of both the PAH and the SPS solutions was adjusted to 4.0, and both solutions contained 0.1 M NaCl. After the first PAH/SPS block, the PAH/PAA block, which consisted of either 10 or 16 layers, was constructed on top (PAA as the first layer). For this block, the PAA solution was adjusted to pH 3.5 and the PAH solution was adjusted to pH 8.5. Afterwards alternating blocks of PAH/SPS (100 layers) and PAH/PAA (10 or 16 layers) were constructed so that the film contained 3, 7, or 11 total blocks. The PAH/PAA blocks were made porous by treatment in a pH 2.3 or pH 2.4 aqueous bath and then subsequent treatment in pH 5.5 water. Heating the films to 180° C. for 2 hours locked in the structure. The cytochalasin D was wicked into the films in either a 0.2 mg/mL or 1.0 mg/mL DMSO solution.

The release of this compound was studied by monitoring the release into a buffer solution using UV-Vis spectroscopy to observe the change of absorbance of the solution with time. The loaded films showed a linear release profile over a span of one month. The effects of cytochalasin D on murine wild type (WT) NR6 fibroblasts was studied by growing the cells on top of the loaded films. The WT NR6 fibroblasts were cultured in pH 7.4 modified Eagles medium-α (MEM-α) with 7.5% (v/v) fetal bovine serum (FBS), 1% (v/v) sodium pyruvate (100 mM), 1% (v/v) nonessential amino acids (10 mM), 1% (v/v) Geneticin (G418) antibiotic (350 μg/10 mL PBS), 1% (v/v) L-glutamine (200 mM), 1% (v/v) penicillin (10,000 U/mL), and 1% streptomycin (10 mg/mL). Cells were kept in a humid incubator at 37.5° C. and 5% CO₂. Cytochalasin D inhibits cells from dividing during mitosis, which results in multinucleated cells with different morphology from normal ones. The cells were affected by the cytochalasin D, meaning it was successfully delivered to them, as confirmed by optical microscopy.

Loading and Releasing of Ketoprofen

Ketoprofen was loaded into porous multilayer films by wicking it up in a DMSO solution. The films were constructed in the same manner as described in the cytochalasin D example above. The ketoprofen was wicked into the films in either a 0.2 mg/mL or 10 mg/mL DMSO solution. The release of this compound was followed by using UV-Vis spectroscopy to observe the change of absorbance of the solution with time. Like cytochalasin D, a linear release was observed.

Loading and Releasing of Other Compounds

A variety of other compounds, including hormones, antibiotics, anti-inflammatory and anti-tumor compounds, were loaded in porous multilayer films. Beta-estradiol, progesterone, camptothecin, indomethacin, ampicillin, and a mixture of penicillin, neomycin and streptomycin were each wicked into the films in either a 0.2 mg/mL or 10 mg/mL DMSO solution. The compounds are released from the films. Films that release antibiotics kill bacteria, such as E. coli or S. epidermis.

Ionic Liquids

Other applications of loaded porous multilayer films are solar cells and batteries made by loading ionic liquids into the pores. The conductive properties of ionic liquids make them a very attractive research topic, but their physical properties make them difficult to work with since they often leak out of devices. After wicking the ionic liquid, 1-ethyl-3-methyl imidazolium trifluromethane sulfonate, into the porous multilayer film, the multilayers acted as a container for the ionic liquid and did not allow it to escape. The conductivity of the device was about 10⁻⁵ S/cm, which is within an order of magnitude of the original liquid ionic conductivity of 10⁻⁴ S/cm. We can control this conductivity by changing the pore density with different pH conditions as explained by percolation theory. This technique could have an immense impact on electro-optical as well as energy storage devices.

Other embodiments are within the scope of the following claims.

What is claimed is:

1. A polymer-containing structure comprising a layer over a substrate, wherein the layer includes a first polymer region and a second polymer region, wherein the first polymer region responds to a greater extent than the second polymer region when exposed to a pore-altering medium.

2. The structure of claim 1, wherein the first region includes a first polyelectrolyte and a second polyelectrolyte.

3. The structure of claim 1, wherein the second region includes a third polyelectrolyte and a fourth polyelectrolyte.

4. The structure of claim 2, wherein the second region includes a third polyelectrolyte and a fourth polyelectrolyte.

5. The structure of claim 1, wherein the first region is disposed between the substrate and the second region.

6. The structure of claim 1, wherein the second region is disposed between the substrate and the first region.

7. The structure of claim 1, wherein the second region remains substantially non-porous when exposed to a pore-altering medium.

8. The structure of claim 1, wherein the first region is porous and the second region is substantially non-porous.

9. The structure of claim 8, wherein the first region includes a nanopore.

10. The structure of claim 9, wherein the first region is substantially free of micropores.

11. The structure of claim 8, wherein the first region becomes less porous when exposed to a pore-closing medium.

12. The structure of claim 8, further comprising a compound in the first region.

13. The structure of claim 12, wherein the compound is a drug.

14. The structure of claim 12, wherein the compound is a liquid crystal.

15. The structure of claim 12, wherein the compound is an ionic liquid.

16. The structure of claim 1, wherein the substrate is glass, plastic or metal.

17. A polymer-containing structure comprising a plurality of alternating fixed and variable regions disposed on a substrate, wherein the variable regions respond to a greater extent than the fixed regions when exposed to a pore-altering medium.

18. The structure of claim 17, wherein a variable region includes a first polyelectrolyte and a second polyelectrolyte.

19. The structure of claim 17, wherein a fixed region includes a third polyelectrolyte and a fourth polyelectrolyte.

20. The structure of claim 17, wherein the variable regions each independently have a thickness less than 1000 nm.

21. The structure of claim 20, wherein the fixed regions each independently have a thickness less than 1000 nm.

22. The structure of claim 21, wherein the alternating fixed and variable regions are arranged as a series of alternating layers over the substrate.

23. The structure of claim 22, wherein the thicknesses and refractive indices of the variable regions and the thicknesses and refractive indices of the fixed regions are selected such that the structure reflects a predetermined wavelength of light.

24. The structure of claim 22, wherein the structure is substantially free of pores having a diameter of 150 micrometers or greater.

25. The structure of claim 21, wherein the thickness of a variable region changes upon exposure to a pore-altering medium.

26. The structure of claim 21, wherein the refractive index of a variable region changes upon exposure to a pore-altering medium.

27. A polymer-containing structure comprising a first polymer region and a second polymer region, wherein the first polymer region has a refractive index that can be altered by an aqueous treatment of the structure.

28. The structure of claim 27, wherein the aqueous treatment alters the porosity of the first region.

29. The structure of claim 28, wherein the aqueous treatment does not substantially alter the porosity of the second region.

30. The structure of claim 27, wherein the aqueous treatment increases the porosity of the first region.

31. The structure of claim 27, wherein the aqueous treatment decreases the porosity of the first region.

32. A drug delivery device comprising a plurality of alternating porous and substantially non-porous regions disposed on a substrate.

33. The device of claim 32, further comprising a compound distributed in a porous region.

34. The device of claim 32, wherein a porous region includes a first polyelectrolyte and a second polyelectrolyte.

35. The device of claim 32, wherein a substantially non-porous region includes a third polyelectrolyte and a fourth polyelectrolyte.

36. The device of claim 32, wherein the plurality of alternating porous and substantially non-porous regions are arranged as a series of alternating layers over the substrate.

37. The device of claim 36, wherein the alternating layers of the series each independently have a thickness and a refractive index selected such that the device reflects light of a predetermined wavelength.

38. The device of claim 37, further comprising a delivery medium distributed in a porous region.

39. The device of claim 38, wherein the delivery medium alters the refractive index of the porous region, thereby altering the reflection of light of a predetermined wavelength.

40. The device of claim 38, wherein the delivery medium includes a drug.

41. The device of claim 38, wherein the device changes color when the delivery medium exits the porous region.

42. An optical device comprising a porous polymeric structure arranged on a substrate and a liquid crystal distributed in the pores of the polymeric structure.

43. The device of claim 42, wherein the porous polymeric structure forms a layer over the substrate.

44. The device of claim 43, further comprising electrodes arranged to apply an electric field across the layer when a voltage is applied to the electrodes.

45. The device of claim 44, wherein applying an electric field across the layer increases transmission of light through the porous polymeric structure.
